(19) United States
(12) Patent Application Publication
(10) Pub. No.: US 2006/0216737 A1
(43) Pub. Date: Sep. 28, 2006

(54) Methods for multiplex amplification

(76) Inventors: John Bodeau, San Mateo, CA (US); Stephen Gunstream, San Francisco, CA (US); Mark Oldham, Los Gatos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/372,242

(22) Filed: Mar. 8, 2006

Related U.S. Application Data
(60) Provisional application No. 60/661,139, filed on Mar. 10, 2005.

(51) Int. Cl. C12Q 1/68; C12P 19/34

(52) U.S. Cl. 435/6; 435/91.2

(57) ABSTRACT

Methods for multiplex amplification of target nucleic acid sequences are provided.

This application claims the benefit of U.S. Provisional Application No. 60/661,139, filed Mar. 10, 2005, which is incorporated by reference herein in its entirety for any purpose.

### FIELD OF THE INVENTION

Methods for multiplex amplification of target nucleic acid sequences are provided.

### BACKGROUND

In certain multiplex amplification methods, multiple sets of primers are used in the same reaction to amplify more than one target nucleic acid sequence. In certain instances, the results of multiplex amplification can suffer when one of the target nucleic acid sequences to be amplified in the reaction is more abundant than another to be amplified in the same reaction. In certain instances, amplification of the more abundant template can competitively interfere with amplification of the less abundant template.

### SUMMARY OF CERTAIN EMBODIMENTS

In certain embodiments, a method of amplifying at least two different target nucleic acid sequences in a sample is provided comprising: forming a plurality of different reaction compositions that each comprise a portion of the sample and at least two primer sets, wherein at least two of the primer sets are specific for a set of at least two different target nucleic acid sequences that are predicted to be present in similar abundance in the sample, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and subjecting the plurality of different reaction compositions to at least one amplification reaction to amplify the sets of different target nucleic acid sequences.

In certain embodiments, a method of amplifying at least two different target nucleic acid sequences in a sample is provided comprising: forming a plurality of different reaction compositions that each comprise a portion of the sample and at least two primer sets, wherein at least two of the primer sets are specific for a set of at least two different target nucleic acid sequences that are present in similar abundance in the sample, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and subjecting the plurality of different reaction compositions to at least one amplification reaction to amplify the sets of different target nucleic acid sequences.

In certain embodiments, a method of amplifying at least two different target nucleic acid sequences in a sample is provided comprising: forming a plurality of different reaction compositions that each comprise a portion of the sample and at least two primer sets, wherein at least two of the primer sets are specific for a set of at least two different target nucleic acid sequences that are present at a copy number within 1000-fold of one another, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and subjecting the plurality of different reaction compositions to at least one amplification reaction to amplify the sets of different target nucleic acid sequences.

### BRIEF DESCRIPTION OF THE DRAWINGS

FIGS. 1A to 1C show the effect of enhanced Master Mix on multiplex polymerase chain reaction (“PCR”) of target nucleic acid sequences of varying abundance in the same reaction, as described in Example 1. FIG. 1A shows a graph of cycle number versus fluorescent intensity (ΔRn) for a singleplex PCR using IL-18 template and Applied Biosystems Universal Master Mix. FIG. 1B shows a graph of cycle number versus fluorescent intensity (ΔRn) for several multiplex PCR reactions in which the concentration of GAPDH template remained constant and the concentration of IL-18 template was varied, using Applied Biosystems Universal Master Mix. FIG. 1C shows a graph of cycle number versus fluorescent intensity (ΔRn) for several multiplex PCR reactions in which the concentration of GAPDH template remained constant and the concentration of IL-18 template was varied, using enhanced Master Mix.

FIGS. 2A and 2B show graphs of cycle number versus fluorescent intensity (ΔRn) for several multiplex PCR reactions in which the target nucleic acid sequences were matched in abundance (FIG. 2A) or mismatched in abundance (FIG. 2B), as described in Example 2.

FIGS. 3A and 3B show graphs of cycle number versus fluorescent intensity (ΔRn) for several multiplex PCR reactions incorporating “enhanced” master mix, in which the templates were matched in abundance (FIG. 3A) or mismatched in abundance (FIG. 3B), as described in Example 3.

### DETAILED DESCRIPTION OF CERTAIN EXEMPLARY EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the event that one or more of the incorporated literature and similar materials defines a term that contradicts that term's definition in this application, this application controls.

Certain Definitions and Terms

The term “nucleotide base” refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases, e.g., adenine, guanine, cytosine, uracil, and thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (61A), N6-Δ2-isopentenyl-2-methylthioadenine (2 ms61A), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide” refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, OR, —NR₂ or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C₁-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226). Exemplary LNA sugar analogs within a polynucleotide include, but are not limited to, the structures: 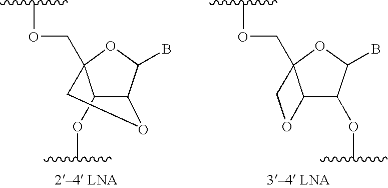
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 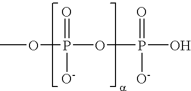
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

The term “nucleotide analog” refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. A polynucleotide may comprise one or more lesions. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Polynucleotides may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 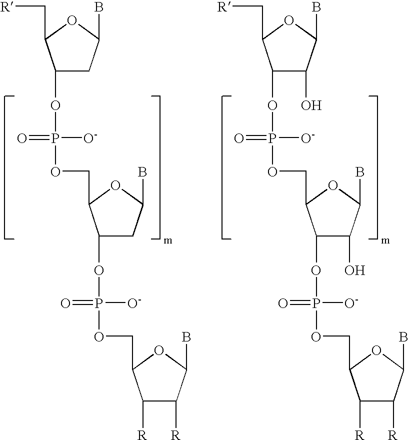
wherein each B is independently the base moiety of a nucleotide, e.g., a purine, a 7-deazapurine, a pyrimidine, or an analog thereof; each m defines the length of the respective nucleic acid and can range from zero to thousands, tens of thousands, or even more; each R is independently selected from the group comprising hydrogen, hydroxyl, halogen, —R″, —OR″, and —NR″R″, where each R″ is independently (C₁-C₆) alkyl or (C5-C1₄) aryl, or two adjacent Rs may be taken together to form a bond such that the ribose sugar is 2′,3′-didehydroribose, and each R′ may be independently hydroxyl or 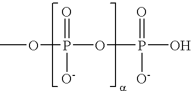
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably, and refer to a polynucleotide that contains at least one nucleotide analog and/or at least one phosphate ester analog and/or at least one pentose sugar analog. A polynucleotide analog may comprise one or more lesions. Also included within the definition of polynucleotide analogs are polynucleotides in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. Base-stacking and hydrophobic interactions may also contribute to duplex stability.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, and target-specific portions.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, and target-specific portions. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 70% of the particular identical sequences. In certain embodiments, “a substantial portion of the particular identical sequences” means at least 80% of the particular identical sequences. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, the more complex the composition, the more likely undesired sequences will hybridize. For example, in certain embodiments, with a given number of mismatches, a probe may more likely hybridize to undesired sequences in a composition with the entire genomic DNA than in a composition with fewer DNA sequences, when the same hybridization and wash conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, and target-specific portions.

The term “primer” refers to a polynucleotide or oligonucleotide that has a free 3′-OH (or functional equivalent thereof) that can be extended by at least one nucleotide in a primer extension reaction catalyzed by a polymerase. In certain embodiments, primers may be of virtually any length, provided they are sufficiently long to hybridize to a target nucleic acid sequence of interest in the environment in which primer extension is to take place. In certain embodiments, primers are specific for a particular target nucleic acid sequence. In certain embodiments, primers are degenerate, e.g., specific for a set of target nucleic acid sequences.

The terms “primer set” or “set of primers” refer to two or more primers that are used as a set. In certain embodiments, a primer set may be designed to hybridize to sequences that flank a specific target nucleic acid sequence to be amplified. In certain embodiments, a primer set may be designed to hybridize to sequences that flank more than one different target nucleic acid sequence to be amplified.

The term “polymerase” refers to an enzyme that is capable of adding at least one nucleotide onto the 3′ end of a primer, or to a primer extension product, that is annealed to a target nucleic acid sequence. In certain embodiments, the nucleotide is added to the 3′ end of the primer in a template-directed manner. In certain embodiments, the polymerase is capable of sequentially adding two or more nucleotides onto the 3′ end of the primer. A “DNA polymerase” catalyzes the polymerization of deoxynucleotides.

The term “thermostable polymerase” refers to a polymerase that retains its ability to add at least one nucleotide onto the 3′ end of a primer, or to a primer extension product, that is annealed to a target nucleic acid sequence at a temperature higher than 37° C. The term “non-thermostable polymerase” refers to a polymerase that does not retain its ability to add at least one nucleotide onto the 3′ end of a primer, or to a primer extension product, that is annealed to a target nucleic acid sequence at a temperature higher than 37° C.

The terms “primer extension” and “primer extension reaction” are used interchangeably, and refer to a process of adding one or more nucleotides to a nucleic acid primer, or to a primer extension product, using a polymerase, a target nucleic acid sequence, and one or more nucleotides.

A “primer extension product” is produced when one or more nucleotides have been added to a primer, or to a primer extension product, in a primer extension reaction. In certain embodiments, a primer extension product serves as a target nucleic acid sequence in subsequent primer extension reactions. In certain embodiments, a primer extension product includes a terminator. In certain embodiments, when a primer extension product includes a terminator, it is referred to as a “primer extension product comprising a terminator.”

A “target nucleic acid sequence” is a sequence in a sample that is not a known control gene that is added to the sample. In certain embodiments, a target nucleic acid sequence serves as a template for amplification in a PCR reaction. In certain embodiments, a target nucleic acid sequence is a portion of a larger nucleic acid sequence. In certain embodiments, a target nucleic acid sequence is a portion of a gene.

“Target nucleic acid sequences that are predicted to be in similar abundance in a sample” means that the number of copies of such target nucleic acid sequences in the sample are predicted to be similar. In certain embodiments, target nucleic acid sequences are considered to be in similar abundance if the number of copies of such sequences varies by as much as five to ten-fold. In certain embodiments, target nucleic acid sequences are considered to be in similar abundance if the number of copies of such sequences varies by as much as 10 to 100-fold. In certain embodiments, target nucleic acid sequences are considered to be in similar abundance if the number of copies of such sequences varies by no more than between 100 to 1000-fold.

As used herein, a “buffering agent” is a compound added to an amplification reaction which modifies the stability, activity, or longevity of one or more components of the amplification reaction by regulating the pH of the amplification reaction. Certain buffering agents are well known in the art and include, but are not limited to, Tris and Tricine.

An “additive” is a compound added to a composition which modifies the stability, activity, or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, dimethylsulfoxide (“DMSO”), glycerol, ethylene glycol, dithiothreitol (“DTT”), Thermoplasma acidophilum inorganic pyrophosphatase (“TAP”), betaine, bovine serum albumin (BSA), propylene glycol, glycinamide, CHES, Percoll, aurintricarboxylic acid, Tween-20, Tween 21, Tween 40, Tween 60, Tween 85, Brij 30, NP-40, Triton X-100, CHAPS, CHAPSO, Mackernium, LDAO, Zwittergent 3-10, Zwittergent 3-14, Zwittergent SB 3-16, Empigen, NDSB-20, pyroPOase, T4G32, E. coli SSB, RecA, nicking endonucleases, 7-deazaG, dUTP and UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction.

A “probe” is a polynucleotide that is capable of binding to a complementary target nucleic acid sequence. In certain embodiments, the probe is used to detect amplified target nucleic acid sequences. In certain embodiments, the probe incorporates a label.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. In certain embodiments, the label emits a detectable signal only when the probe is bound to a complementary target nucleic acid sequence. In certain embodiments, the label emits a detectable signal only when the label is cleaved from the polynucleotide probe. In certain embodiments, the label emits a detectable signal only when the label is cleaved from the polynucleotide probe by a 5′ exonuclease reaction.

Labels may be “detectably different”, which means that they are distinguishable from one another by at least one detection method. Detectably different labels include, but are not limited to, labels that emit light of different wavelengths, labels that absorb light of different wavelengths, labels that scatter light of different wavelengths, labels that have different fluorescent decay lifetimes, labels that have different spectral signatures, labels that have different radioactive decay properties, labels of different charge, and labels of different size. In certain embodiments, the label emits a fluorescent signal.

“Endpoint polymerase chain reaction” or “endpoint PCR” is a polymerase chain reaction method in which the presence or quantity of nucleic acid target sequence is detected after the PCR reaction is complete, and not while the reaction is ongoing.

“Real-time polymerase chain reaction” or “real-time PCR” is a polymerase chain reaction method in which the presence or quantity of nucleic acid target sequence is detected while the reaction is ongoing. In certain embodiments, the signal emitted by one or more probes present in a reaction composition is monitored during each cycle of the polymerase chain reaction as an indicator of synthesis of a primer extension product. In certain embodiments, fluorescence emitted during each cycle of the polymerase chain reaction is monitored as an indicator of synthesis of a primer extension product.

A “multiplex amplification reaction” is an amplification reaction in which two or more target nucleic acid sequences are amplified in the same reaction. A “multiplex polymerase chain reaction” or “multiplex PCR” is a polymerase chain reaction method in which two or more target nucleic acid sequences are amplified in the same reaction.

A “singleplex amplification reaction” is an amplification reaction in which only one target nucleic acid sequence is amplified in the reaction. A “singleplex polymerase chain reaction” or “singleplex PCR” is a polymerase chain reaction method in which only one target nucleic acid sequence is amplified in the reaction.

The term “treatment” refers to the process of subjecting one or more cells, cell lines, tissues, or organisms to a condition, substance, or agent (or combination thereof) that may cause the cell, cell line, tissue, or organism to alter its gene expression profile. In certain embodiments, a treatment may include a range of chemical concentrations and exposure times, and replicate samples may be generated. The term “untreated control” refers to a sample obtained from a cell, cell line, tissue, or organism that has not been exposed to a treatment.

“Threshold cycle” or “CT” is defined as the cycle number at which the observed signal from a target nucleic acid sequence-specific probe exceeds a fixed threshold. In certain embodiments, the fixed threshold is set as the amount of signal observed in a reaction lacking a target nucleic acid sequence. In certain embodiments, the fixed threshold is set at a level above the background noise signal. In certain such embodiments, the fixed threshold is set at a value corresponding to 3 or more times the combination of the root mean squared of the background noise signal and the background noise signal. In certain embodiments, the observed signal is from a fluorescent label.

The term “amplification bias” refers to the efficiency with which at least one primer set amplifies certain nucleic acids compared to certain other different nucleic acids. In certain instances, individual target nucleic acid sequences of a plurality of different target nucleic acid sequences amplified by at least one primer set will not be amplified by the same amount. In other words, in certain instances, amplification of certain target nucleic acid sequences will be favored over amplification of certain other different target nucleic acid sequences. Thus, in certain such instances, some amplification products from certain target nucleic acid sequences will be more abundant than others after amplification of the target nucleic acid sequences. In certain such instances, the difference in quantity between the different amplification products is the result of amplification bias. For example and not limitation, in certain instances, at least one primer set will preferentially amplify more abundant target nucleic acid sequences compared to less abundant target nucleic acid sequences. In certain instances, the difference in quantity between the different amplification products is the result of reagent depletion. For example and not limitation, in certain instances, amplification of the more abundant target nucleic acid sequence depletes reagent components, thereby terminating amplification before detectable amplification of the less abundant target nucleic acid sequence.

In certain embodiments, the composition of the primer set affects the amplification bias. Thus, in certain embodiments, different primer sets, with different sequences, will have different amplification biases.

In certain embodiments, differences between amplification biases between different primer sets can be seen by examining the amplification profiles of the different primer sets. The term “amplification profile” refers to the results of an analysis of amplification products produced by a set of primers. In certain embodiments, an amplification profile can be determined by quantitating the amplification products comprising a portion or portions of a nucleic acid. In certain embodiments, an amplification profile is determined by quantitating the amplification products comprising two or more portions.

For example and not limitation, where a first primer set and a second primer set are used to amplify the same plurality of target nucleic acid sequences under the same conditions, the first primer set may produce more amplification product comprising a first portion than the second primer set. That second primer set may, however, produce more amplification product comprising a second portion than the first primer set. Thus, the first primer set has a different amplification profile from the second primer set. In certain embodiments, a third primer set may produce more amplification product comprising the first portion and amplification product comprising the second portion than either the first primer set or the second primer set. That third primer set would have a different amplification profile than either the first primer set or the second primer set. In certain embodiments, each primer set has a distinct amplification profile.

Certain Exemplary Components

Target Nucleic Acid Sequences

In certain embodiments, target nucleic acid sequences include RNA and DNA. Exemplary RNA target sequences include, but are not limited to, mRNA, rRNA, tRNA, snRNA, viral RNA, and variants of RNA, such as splicing variants. Exemplary DNA target sequences include, but are not limited to, genomic DNA, plasmid DNA, phage DNA, nucleolar DNA, mitochondrial DNA, chloroplast DNA, cDNA, synthetic DNA, yeast artificial chromosomal DNA (“YAC”), bacterial artificial chromosome DNA (“BAC”), other extrachromosomal DNA, and primer extension products. Target nucleic acid sequences also include, but are not limited to, analogs of both RNA and DNA. Exemplary nucleic acid analogs include, but are not limited to, locked nucleic acids (“LNAs”), peptide nucleic acids (“PNAs”), 8-aza-7-deazaguanine (“PPG's”), and other nucleic acid analogs. Exemplary target nucleic acid sequences include, but are not limited to, chimeras of RNA and DNA.

A variety of methods are available for obtaining a target nucleic acid sequence. When the nucleic acid target is obtained through isolation from a biological matrix, certain isolation techniques include, but are not limited to, (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), in certain embodiments, using an automated nucleic acid extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced nucleic acid precipitation methods (e.g., Miller et al., Nucleic Acids Research, 16(3): 9-10 (1988)), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613.

In certain embodiments, a target nucleic acid sequence may be derived from any living, or once living, organism, including but not limited to, a prokaryote, a eukaryote, a plant, an animal, and a virus. In certain embodiments, a target nucleic acid sequence is derived from a human. In certain embodiments, the target nucleic acid sequence may originate from a nucleus of a cell, e.g., genomic DNA, or may be extranuclear nucleic acid, e.g., originate from a plasmid, a mitochondrial nucleic acid, from various RNAs, and the like. In certain embodiments, if the sequence from the organism is RNA, it may be reverse-transcribed into a cDNA target nucleic acid sequence. In certain embodiments, the target nucleic acid sequence may be present in a double-stranded or single-stranded form.

In certain embodiments, multiple target nucleic acid sequences can be amplified in the same reaction (e.g., in multiplex amplification reactions). In certain embodiments, more than one different multiplex amplification reaction is performed. In certain embodiments, 5 to 10 different multiplex amplification reactions are performed. In certain embodiments, 10 to 25 different multiplex amplification reactions are performed. In certain embodiments, 25 to 50 different multiplex amplification reactions are performed. In certain embodiments, greater than 50 different multiplex amplification reactions are performed.

In certain embodiments, a sufficient number of different amplification reactions can be performed such that all of the target nucleic acid sequences together represent all of the genes in a genome. In certain embodiments, the genome may be derived from any living, or once living organism including but not limited to, a prokaryote, a eukaryote, a plant, an animal, and a virus. In certain embodiments, the genome is human. In certain embodiments, a sufficient number of different amplification reactions can be performed such that all of the target nucleic acid sequences together represent most of the genes in a genome. In certain embodiments, a sufficient number of different amplification reactions can be performed such that all of the target nucleic acid sequences together represent all of the nucleic acids in a transcriptome. In certain embodiments, a sufficient number of different amplification reactions can be performed such that all of the target nucleic acid sequences together represent most of the nucleic acids in a transcriptome. The term “transcriptome” refers to the activated genes, mRNAs, and/or transcripts found in a particular tissue at a particular time.

Exemplary target nucleic acid sequences include, but are not limited to, amplification products, ligation products, transcription products, reverse transcription products, primer extension products, methylated DNA, and cleavage products. Exemplary amplification products include, but are not limited to, PCR and isothermal products.

Different target nucleic acid sequences may be different portions of a single contiguous nucleic acid or may be on different nucleic acids. Different portions of a single contiguous nucleic acid may or may not overlap.

In certain embodiments, nucleic acids in a sample may be subjected to a cleavage procedure. In certain embodiments, such cleavage products may be target nucleic acid sequences.

In certain embodiments, a target nucleic acid sequence is derived from a crude cell lysate. Examples of target nucleic acid sequences include, but are not limited to, nucleic acids from buccal swabs, crude bacterial lysates, blood, skin, semen, hair, bone, mucus, saliva, cell cultures, and tissue biopsies.

In certain embodiments, target nucleic acid sequences are obtained from a cell, cell line, tissue, or organism that has undergone a treatment. In certain embodiments, the treatment results in the up-regulation or down-regulation of certain target nucleic acid sequences in treated cells, cell lines, tissues, or organisms.

In certain embodiments, a target nucleic acid sequence is obtained from a single cell. In certain embodiments, a target nucleic acid sequence is obtained from tens of cells. In certain embodiments, a target nucleic acid sequence is extracted from hundreds of cells or more. In certain embodiments, a target nucleic acid sequence is extracted from cells of a single organism. In certain embodiments, a target nucleic acid sequence is extracted from cells of two or more different organisms. In certain embodiments, a target nucleic acid sequence concentration in a PCR reaction ranges from about 1 to about 10,000,000 molecules per reaction.

Primers

In certain embodiments, each primer is sufficiently long to prime the template-directed synthesis of the target nucleic acid sequence under the conditions of the amplification reaction. In certain embodiments, the lengths of the primers depends on many factors, including, but not limited to, the desired hybridization temperature between the primers, the target nucleic acid sequence and the complexity of the different target nucleic acid sequences to be amplified, and other factors. In certain embodiments, a primer is about 15 to about 35 nucleotides in length. In certain embodiments, a primer is fewer than 15 nucleotides in length. In certain embodiments, a primer is greater than 35 nucleotides in length.

In certain embodiments, a set of primers comprises at least one set of primers which comprises at least one designed portion and at least one random portion. In certain embodiments, the designed portion of a primer set is at the 5′ end of the primers. In certain embodiments, the designed portion of a primer set is at the 3′ end of the primers. In certain embodiments, the designed portion of a primer set is in the center of the primers. In certain embodiments, the designed portion of a primer set includes two or more designed portions. In certain embodiments, the designed portions of a primer set are located in two or more portions separated by random portions.

Probes and Labels

In certain embodiments, a probe may include Watson-Crick bases or modified bases. Modified bases include, but are not limited to, the AEGIS bases (from Eragen Biosciences), which have been described, e.g., in U.S. Pat. Nos. 5,432,272; 5,965,364; and 6,001,983. In certain embodiments, bases are joined by a natural phosphodiester bond or a different chemical linkage. Different chemical linkages include, but are not limited to, a peptide bond or an LNA linkage, which is described, e.g., in published PCT applications WO 00/56748; and WO 00/66604.

In certain embodiments, oligonucleotide probes present in a multiplex amplification are suitable for monitoring the amount of amplification product produced as a function of time. Such oligonucleotide probes include, but are not limited to, the 5′-exonuclease assay (TaqMan) probes (see above and also U.S. Pat. No. 5,538,848), stem-loop molecular beacons (see, e.g., U.S. Pat. Nos. 6,103,476 and 5,925,517 and Tyagi & Kramer, 1996, Nature Biotechnology 14:303-308), stemless or linear beacons (see, e.g., WO 99/21881), PNA Molecular Beacons (see, e.g., U.S. Pat. Nos. 6,355,421 and 6,593,091), linear PNA beacons (see, e.g. Kubista et al., 2001, SPIE 4264:53-58), non-FRET probes (see, e.g., U.S. Pat. No. 6,150,097), Sunrise®™/Amplifluor®™ probes (see, e.g., U.S. Pat. No. 6,548,250), stem-loop and duplex Scorpion™ probes (see, e.g., Solinas et al., 2001, Nucleic Acids res. 29: E96 and U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nat. Biotechnol. 17:804-807; Isacsson et al., 2000, Mol. Cell. Probes. 14:321-328; Svanvik et al., 2000, Anal Biochem. 281:26-35; Wolffs et al., 2001, Biotechniques 766:769-771; Tsourkas et al., 2002, Nucleic Acids Res. 30:4208-4215; Riccelli et al., 2002, Nucleic Acids Res. 30:4088-4093; Zhang et al., 2002, Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc. 14:11155-11161.

In certain embodiments, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody/antigen, ionic complexes, hapten/ligand, e.g., biotin/avidin. In certain embodiments, use of labels can be accomplished using any one of a large number of known techniques employing known labels, linkages, linking groups, reagents, reaction conditions, and analysis and purification methods.

Labels include, but are not limited to, light-emitting, light-scattering, and light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28, and Non-Radioactive Labelling, A Practical Introduction, Garman, A. J. (1997) Academic Press, San Diego). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. In certain embodiments, the fluorescent label is selected from SYBR®-green, 6-carboxyfluorescein (“FAM”), TET, ROX, VIC™, and JOE. In certain embodiments, a label is a radiolabel.

In certain embodiments, labels are hybridization-stabilizing moieties which serve to enhance, stabilize, or influence hybridization of duplexes, e.g. intercalators and intercalating dyes (including, but not limited to, ethidium bromide and SYBR® green), minor-groove binders, and cross-linking functional groups (see, e.g., Blackburn, G. and Gait, M. Eds. “DNA and RNA structure” in Nucleic Acids in Chemistry and Biology, 2^{nd} Edition, (1996) Oxford University Press, pp. 15-81). In certain embodiments, labels effect the separation or immobilization of a molecule by specific or non-specific capture, for example biotin, digoxigenin, and other haptens (see, e.g., Andrus, A. “Chemical methods for 5′ non-isotopic labeling of PCR probes and primers” (1995) in PCR 2: A Practical Approach, Oxford University Press, Oxford, pp. 39-54).

In certain embodiments, different probes comprise detectable and different labels that are distinguishable from one another. For example, in certain embodiments, labels are different fluorophores capable of emitting light at different, spectrally-resolvable wavelengths (e.g., 4-differently colored fluorophores); certain such labeled probes are known in the art and described above, and in, e.g., U.S. Pat. No. 6,140,054 and Saiki et al., 1986, Nature 324:163-166.

In certain embodiments, one or more of the primers in an amplification reaction acts as a probe. In certain embodiments, one or more of the primers in an amplification reaction includes a label.

Polymerases

In certain embodiments, a polymerase is active at 37° C. In certain embodiments, a polymerase is active at a temperature other than 37° C. In certain embodiments, a polymerase is active at a temperature greater than 37° C. In certain embodiments, a polymerase is active at both 37° C. and other temperatures.

In certain embodiments, a thermostable polymerase remains active at a temperature greater than about 42° C. In certain embodiments, a thermostable polymerase remains active at a temperature greater than about 50° C. In certain embodiments, a thermostable polymerase remains active at a temperature greater than about 60° C. In certain embodiments, a thermostable polymerase remains active at a temperature greater than about 70° C. In certain embodiments, a thermostable polymerase remains active at a temperature greater than about 80° C. In certain embodiments, a thermostable polymerase remains active at a temperature greater than about 90° C.

Exemplary thermostable polymerases include, but are not limited to, Thermus thermophilus HB8 (described, e.g., in U.S. Pat. No. 5,789,224); mutant Thermus thermophilus HB8, including, but not limited to, Thermus thermophilus HB8 (D18A; F669Y; E683R), Thermus thermophilus HB8 (A271; F669Y; E683W), and Thermus thermophilus HB8 (D18A; F669Y); Thermus oshimai (described, e.g., in U.S. Provisional Application No. 60/334,798, filed Nov. 30, 2001, corresponding to U.S. Application No. 20030194726, Thermus oshimai Nucleic Acid Polymerases, published Oct. 16, 2003); mutant Thermus oshimai, including, but not limited to, Thermus oshimai (G43D; F665Y); Thermus scotoductus (described, e.g., in U.S. Provisional Application No. 60/334,489, filed Nov. 30, 2001); mutant Thermus scotoductus, including, but not limited to, Thermus scotoductus (G46D; F668Y); Thermus thermophilus 1B21 (described, e.g., in U.S. Provisional Application No. 60/336,046, filed Nov. 30, 2001), mutant Thermus thermophilus 1B21, including, but not limited to, Thermus thermophilus 1B21 (G46D; F669Y); Thermus thermophilus GK24 (described, e.g., in U.S. Provisional Application No. 60/336,046, filed Nov. 30, 2001); mutant Thermus thermophilus GK24, including, but not limited to, Thermus thermophilus GK24 (G46D; F669Y); Thermus aquaticus polymerase; mutant Thermus aquaticus polymerase, including, but not limited to, Thermus aquaticus (G46D; F667Y) (AmpliTaq® FS or Taq (G46D; F667Y), described, e.g., in U.S. Pat. No. 5,614,365), Taq (G46D; F667Y; E6811), and Taq (G46D; F667Y; T664N; R660G); Pyrococcus furiosus polymerase; mutant Pyrococcus furiosus polymerase; Thermococcus gorgonarius polymerase; mutant Thermococcus gorgonarius polymerase; Pyrococcus species GB-D polymerase; mutant Pyrococcus species GB-D polymerase; Thermococcus sp. (strain 9°N-7) polymerase; mutant Thermococcus sp. (strain 9°N-7) polymerase; Bacillus stearothermophilus polymerase; mutant Bacillus stearothermophilus polymerase; Tsp polymerase; mutant Tsp polymerase; ThermalAce™ polymerase (Invitrogen); Thermus flavus polymerase; mutant Thermus flavus polymerase; Thermus litoralis polymerase; mutant Thermus litoralis polymerase. In certain embodiments, a thermostable polymerase is a mutant of a naturally-occurring polymerase.

Exemplary non-thermostable polymerases include, but are not limited to DNA polymerase I; mutant DNA polymerase I, including, but not limited to, Klenow fragment and Klenow fragment (3′→5′ exonuclease minus); T4 DNA polymerase; mutant T4 DNA polymerase; T7 DNA polymerase; mutant T7 DNA polymerase; phi29 DNA polymerase; and mutant phi29 DNA polymerase.

In certain embodiments, a polymerase is a processive polymerase. In certain embodiments, a processive polymerase remains associated with the template for two or more nucleotide additions. In certain embodiments, a non-processive polymerase disassociates from the template after the addition of each nucleotide. In certain embodiments, a processive DNA polymerase has a characteristic polymerization rate. In certain embodiments, a processive DNA polymerase has a polymerization rate of between 5 to 300 nucleotides per second. In certain embodiments, a processive DNA polymerase has a higher processivity in the presence of accessory factors, such as one or more additives. In certain embodiments, the processivity of a processive DNA polymerase may be influenced by the presence or absence of accessory single-stranded DNA-binding proteins and helicases. In certain embodiments, the net polymerization rate will depend on the enzyme concentration, because at higher concentrations there are more re-initiation events and thus the net polymerization rate is increased. In certain embodiments, the processive polymerase is Bst polymerase.

“Strand displacement” as used herein refers to the phenomenon in which a chemical, physical, or biological agent causes at least partial dissociation of a nucleic acid that is hybridized to its complementary strand. In certain embodiments, a DNA polymerase is a strand displacement polymerase. In certain embodiments, a processive DNA polymerase is also a strand displacement polymerase, which is capable of displacing a hybridized strand encountered during replication. In certain embodiments, a strand displacement polymerase requires a factor that facilitates strand displacement to be capable of displacing a hybridized strand encountered during replication. In certain embodiments, a strand displacement polymerase is capable of displacing a hybridized strand encountered during replication in the absence of a strand displacement factor. In certain embodiments, the strand displacement polymerase lacks 5′ to 3′ exonuclease activity.

In certain embodiments, the dissociation of a nucleic acid that is hybridized to its complementary strand occurs in a 5′ to 3′ direction in conjunction with replication. In certain embodiments, where a primer extension reaction forms a newly synthesized strand while displacing a second nucleic acid strand from the template nucleic acid strand, both the newly synthesized and displaced second nucleic acid strand have the same base sequence, which is complementary to the template nucleic acid strand. In certain embodiments, a molecule comprises both strand displacement activity and another activity. In certain embodiments, a molecule comprises both strand displacement activity and polymerase activity. In certain embodiments, strand displacement activity is the only activity associated with a molecule. Enzymes that possess both strand displacement activity and polymerase activity include, but are not limited to, E. coli DNA polymerase 1, the Klenow fragment of DNA polymerase 1, the bacteriophage T7 DNA polymerase, the bacteriophage T5 DNA polymerase, the (p29 polymerase, and the Bst polymerase. Certain methods of using enzymes possessing strand displacement activity include, but are not limited to, those described by, e.g., Kornberg, A., DNA Replication, W.H. Freeman & Co., San Francisco, Calif., 1980.

The term “strand displacement replication” refers to nucleic acid replication which involves strand displacement. In certain embodiments, strand displacement is facilitated through the use of a strand displacement factor, such as a helicase. In certain embodiments, a DNA polymerase that can perform a strand displacement replication in the presence of a strand displacement factor is used in strand displacement replication. In certain embodiments, the DNA polymerase does not perform a strand displacement replication in the absence of such a factor. Strand displacement factors useful in strand displacement replication include, but are not limited to, BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehnan, J. Virology 67(2):711-715 (1993); Skaliter and Lehman, Proc. Natl. Acad. Sci. USA 91(22):10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); and calf thymus helicase (Siegel et al., J. Biol. Chem. 267:13629-13635 (1992)). Strand displacement amplification (SDA) reaction methods include, but are not limited to, those described in, e.g., Fraiser et al., U.S. Pat. No. 5,648,211; Cleuziat et al., U.S. Pat. No. 5,824,517; and Walker et al., Proc. Natl. Acad. Sci. U.S.A. 89:392-396 (1992)).

In certain embodiments, the ability of a polymerase to carry out strand displacement replication can be determined by using the polymerase in a strand displacement replication assay such as those described, e.g., in U.S. Pat. No. 6,642,034, or in a primer-block assay described, e.g., in Kong et al., J. Biol. Chem. 268:1965-1975 (1993).

In certain embodiments, an amplification reaction comprises a blend of polymerases. In certain such embodiments, at least one polymerase possesses exonuclease activity. In certain embodiments, none of the polymerases in an amplification reaction possess exonuclease activity. Exemplary polymerases that may be used in an amplification reaction include, but are not limited to, φ29 DNA polymerase, taq polymerase, stoffel fragment, Bst DNA polymerase. E. coli DNA polymerase 1, the Klenow fragment of DNA polymerase 1, the bacteriophage T7 DNA polymerase, the bacteriophage T5 DNA polymerase, and other polymerases known in the art. In certain embodiments, a polymerase is inactive in the reaction composition and is subsequently activated at a given temperature.

Certain Exemplary Amplification Reaction Methods

In certain embodiments, an amplification reaction composition is formed comprising (a) two or more target nucleic acid sequences, (b) at least one set of primers, and (c) at least one polymerase.

In certain embodiments, an amplification reaction composition is formed comprising two or more target nucleic acid sequences, at least one primer set, dNTPs, at least one buffering agent and at least one polymerase. In certain such embodiments, the amplification reaction is incubated under conditions that allow the formation of one or more amplification products. In certain embodiments, the amplification reaction further includes one or more additives. In certain embodiments, no strand displacement factors are required for strand displacement.

In certain embodiments, an amplification reaction composition comprises strand displacement factors. Exemplary strand displacement factors include, but are not limited to, helicases and single stranded DNA binding protein. In certain embodiments, the temperature of the reaction affects strand displacement. In certain embodiments, a temperature of approximately 40° C., 45° C., 50° C., 55° C., 60° C., 65° C., 70° C., 75° C., 80° C., 85° C., or 90° C. facilitates strand displacement by allowing segments of double stranded DNA to separate and reanneal.

In certain embodiments, the temperature of the amplification reaction is kept at isothermal reaction conditions. The term “isothermal reaction conditions” refers to conditions wherein the temperature is kept substantially constant. In certain embodiments, isothermal reaction conditions prevent the template DNA from being completely disassociated. In certain embodiments, short primers can hybridize to a double stranded template maintained at an isothermal temperature. In certain such embodiments, the primers that strand invade and anneal to the template DNA can be extended by a strand-displacing DNA polymerase. In certain embodiments, an amplification process is isothermal at 50° C. and uses Bst DNA polymerase for strand displacement and extension. In certain embodiments, an amplification reaction uses a fragment of Bst DNA polymerase with the 3′→5′ exonuclease activity removed (“the large fragment of Bst DNA polymerase”).

Certain amplification methods include, but are not limited to, Random PCR or Primer Extension Preamplification-PCR (PEP-PCR) (Zhang et al., Proc. Natl. Acad. Sci., USA 89: 5847-51 (1992)), Linker Adapter PCR (Miyashita et al., Cytogenet. Cell Genet. 66(1): 54-57 (1994)), Tagged-PCR (Grothues et. al., Nuc. Acids Res. 21(5) 1321-1322 (1993)), Inter-Alu-PCR (Bicknell et. al., Genomics 10:186-192 (1991)), Degenerate Oligonucleotide Primed-PCR (DOP-PCR) (Cheung et al., Proc. Natl. Acad. Sci., USA 93:14676-14679 (1996)), Improved-Primer Extension Preamplification PCR (I-PEP-PCR) (Dietmaier et al., Amer. J. Pathology 154(1): 83-95 (1999) and U.S. Pat. No. 6,365,375), LL-DOP PCR (Kittler et al., Anal. Biochem. 300:237-244 (2002)), Balanced PCR amplification (Makrigiorgos et. al., Nature Biotech. 20:936-939 (2002)), Multiple Displacement Amplification (MDA) (U.S. Pat. Nos. 6,124,120 and 6,280,949), and Random Primer Amplification (RPA) (U.S. Pat. No. 5,043,272). In certain embodiments, multiplex amplification may be used (see, e.g., Published U.S. Patent Application No. 2004-0175733 A1).

In certain embodiments, In certain embodiments, multiplex amplification is used to distinguish between target nucleic acid sequences that have single nucleotide polymorphisms (“SNP”). In certain such embodiments, one or more multiplex amplification reactions include one or more primer sets specific for two or more target nucleic acid sequences that differ at only a single nucleotide and are present in similar abundance. In certain such embodiments, the one or more multiplex amplification reactions further include one or more probes with different detectable labels specific for the presence or absence of that particular single nucleotide. In certain such embodiments, the signal from the label in the multiplex amplification reaction is detected as an indicator of the presence of one or more SNPs.

In certain embodiments, multiplex amplification is used for melting curve analysis. In certain such embodiments, a multiplex amplification reaction includes two or more primer sets specific for two or more target nucleic acid sequences of similar abundance and also includes one or more probes that intercalate into double-stranded target nucleic acid sequences and does not bind to single-stranded target nucleic acid sequences. In certain such embodiments, a multiplex amplification reaction includes two or more primer sets specific for two or more target nucleic acid sequences and also includes one or more probes that bind to single-stranded target nucleic acid sequences but does not bind to double-stranded target nucleic acid sequences. In certain such embodiments, the one or more probes includes a detectable label, and the label is detectable only when the one or more probes interact with their target nucleic acid sequences. In certain such embodiments, the temperature of the reaction is modified gradually and the signal from the detectable label is monitored such that the shift of the one or more target nucleic acid sequences from single-stranded to double-stranded or from double-stranded to single-stranded as a function of temperature is recorded. In certain such embodiments, the signal from the detectable label is monitored using real-time PCR.

In certain embodiments, a multiplex amplification reaction includes two or more primer sets specific for two or more target nucleic acid sequences and also includes two or more probes that bind to single-stranded target nucleic acid sequences but do not bind to double-stranded target nucleic acid sequences. In certain embodiments, the two or more probes include a detectable label, and the label is detectable only when the two or more probes interact with their target nucleic acid sequence(s). In certain embodiments, the temperature of the reaction is modified gradually and the signal from the detectable label is monitored such that the shift of one or more target nucleic acid sequences from single-stranded to double stranded or from double-stranded to single-stranded as a function of temperature is recorded. In certain such embodiments, the signal from the detectable label is monitored using real-time PCR.

In certain embodiments, one or more target nucleic acid sequences undergo a treatment before being included in an amplification reaction. In certain embodiments, a target nucleic acid treatment selectively modifies a target nucleic acid according to the methylation state of the target nucleic acid sequence (see, e.g., Published U.S. Patent Application No. 2004-0101843, U.S. Pat. No. 6,265,171; and U.S. Pat. No. 6,331,393). In certain embodiments, the sample from which one or more target nucleic acid sequences is derived (e.g., a cell, tissue, etc.) undergoes a treatment prior to the inclusion of the target nucleic acid sequences from that sample in a multiplex amplification reaction. In certain embodiments, the amplification of one or more target nucleic acid sequences from a treated sample is compared to the amplification of one or more target nucleic acid sequences from an untreated control sample. In certain such embodiments, the expression of one or more genes in response to the treatment is determined.

In certain embodiments, the products of two or more amplification reactions are combined. In certain such embodiments, the products of one amplification reaction may have a different amplification profile than the products of the second amplification reaction.

In various embodiments, amplified target nucleic acid sequences can be used for any purpose for which nucleic acids are used. Certain exemplary uses for amplification products include, but are not limited to, forensic purposes, genotyping, sequencing, detecting SNPs, detecting microsatellite DNA, detecting expression of genes, quantifying expression of genes, nucleic acid library construction, melting curve analysis, and any other purpose that involves manipulating and/or detecting nucleic acids or nucleic acid sequences.

In certain embodiments, amplification products may be used in any process that uses nucleic acids. Exemplary assays in which amplification products may be used include, but are not limited to, agarose gel electrophoresis, picogreen assays, oligonucleotide ligation assays, and assays described in U.S. Pat. Nos. 5,470,705, 5,514,543, 5,580,732, 5,624,800, 5,807,682, 6,759,202, 6,756,204, 6,734,296, 6,395,486, U.S. patent application Ser. Nos. 09/584,905 and 09/724,755, and Published U.S. Patent Application No. US 2003-0190646 A1. In certain embodiments, amplification products are treated before they are used in a downstream process. Such treatments include, but are not limited to, heating or enzymatic digestion of amplification products prior to their use in a downstream process.

In certain embodiments, high-throughput assay systems are used. In certain embodiments, a high-throughput assay system includes a plurality of multiplex amplification reactions. In certain such embodiments, the plurality of multiplex amplification reactions is contained on one or more plates or cards, in separate reaction spaces (including, but not limited to, wells or spots). In certain such embodiments, each of the plurality of multiplex amplification reactions amplifies two to five target nucleic acid sequences of similar abundance. In certain such embodiments, each of the plurality of multiplex amplification reactions amplifies more than five target nucleic acid sequences of similar abundance. In certain such embodiments, each of the plurality of multiplex amplification reactions includes a sufficient number of differently-labeled probes such that the amplification product of each target nucleic acid sequence can be separately identified. In certain such embodiments, the amplification reaction proceeds using real-time PCR.

Exemplary high-throughput assay systems include, but are not limited to, an Applied Biosystems plate-reader system (using a plate with any number of wells, including, but not limited to, a 96-well plate, a-384 well plate, a 768-well plate, a 1,536-well plate, a 3,456-well plate, a 6,144-well plate, and a plate with 30,000 or more wells), the ABI 7900 Micro Fluidic Card system (using a card with any number of wells, including, but not limited to, a 384-well card), other microfluidic systems that exploit the use of TaqMan probes (including, but not limited to, systems described in WO 04083443 A1, and published U.S. Patent Application Nos. 2003-0138829 A1 and 2003-0008308 A1), other micro card systems (including, but not limited to, WO04067175 A1, and published U.S. Patent Application Nos. 2004-083443 A1, 2004-0110275 A1, and 2004-0121364 A1), the Invader® system (Third Wave Technologies), the OpenArray™ system (Biotrove), systems including integrated fluidic circuits (Fluidigm), and other assay systems known in the art. In certain embodiments, multiple different labels are used in each multiplex amplification reaction in a high-throughput multiplex amplification assay system such that a large number of different target nucleic acid sequences can be analyzed on a single plate or card. In certain embodiments, a high-throughput multiplex amplification assay system is capable of analyzing most of the genes in a genome on a single plate or card. In certain embodiments, a high-throughput multiplex amplification assay system is capable of analyzing all genes in an entire genome on a single plate or card. In certain embodiments, a high-throughput multiplex amplification assay system is capable of analyzing most of the nucleic acids in a transcriptome on a single plate or card. In certain embodiments, a high-throughput multiplex amplification assay system is capable of analyzing all of the nucleic acids in a transcriptome on a single plate or card.

When referring to analyzing most of the genes in a genome by performing one or more amplification reactions, for each gene analyzed, either an entire gene may be amplified or a portion of an entire gene may be amplified. When referring to analyzing all of the genes in a genome by performing one or more amplification reactions, for each gene analyzed, either an entire gene may be amplified or a portion of an entire gene may be amplified. When referring to analyzing most of the nucleic acids in a transcriptome by performing one or more amplification reactions, for each nucleic acid analyzed, either an entire nucleic acid or a portion of an entire nucleic acid may be amplified. When referring to analyzing all of the nucleic acids in a transcriptome by performing one or more amplification reactions, for each nucleic acid analyzed, either an entire nucleic acid or a portion of an entire nucleic acid may be amplified.

Certain Exemplary Methods of Multiplex Amplification

Certain available methods to amplify target nucleic acid sequences in a multiplex amplification reaction fail to amplify the target nucleic acid sequences in an even manner, generating a biased amplification product. In certain embodiments, multiplex amplification methods result in a decrease in amplification bias. Certain available methods to amplify target nucleic acid sequences in a multiplex amplification reaction fail to amplify one or more of the target nucleic acid sequences to detectable levels. In certain embodiments, multiplex amplification methods result in a decrease in amplification bias by reducing early depletion of reagents and premature cessation of amplification. In certain embodiments, multiplex amplification methods result in an elimination of amplification bias.

In certain embodiments, a method of amplifying at least two different target nucleic acid sequences in a sample is provided, comprising: forming a plurality of different reaction compositions that each comprise a portion of the sample and at least two primer sets, wherein at least two of the primer sets are specific for a set of at least two different target nucleic acid sequences that are predicted to be present in similar abundance in the sample, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and subjecting the reaction composition to at least one amplification reaction to amplify the set of different target nucleic acid sequences.

In certain embodiments, a method of amplifying at least two different target nucleic acid sequences in a sample is provided, comprising: forming a plurality of different reaction compositions that each comprise a portion of the sample, at least one primer set and at least two probes, wherein the at least one primer set is specific for a set of at least two different target nucleic acid sequences that are predicted to be present in similar abundance in the sample, wherein at least two probes of each of the plurality of different reaction compositions are different from probes in other reaction compositions of the plurality of different reaction compositions, such that different sets of target nucleic acid sequences are detected in different reaction compositions during the at least one amplification reaction; and subjecting the reaction composition to at least one amplification reaction to amplify the set of different target nucleic acid sequences.

In certain embodiments, an amplification reaction is designed so as to amplify only similarly abundant target nucleic acid sequences. In certain embodiments, an amplification reaction is designed so as to amplify only equally abundant target nucleic acid sequences. In certain embodiments, a plurality of target nucleic acid sequences with varied abundance are present in a multiplex amplification reaction, and the multiplex amplification reaction includes at least two primers that are specific for two or more similarly abundant target nucleic acid sequences from the plurality of target nucleic acid sequences. In certain embodiments, a plurality of target nucleic acid sequences with varied abundance are present in a multiplex amplification reaction, and the multiplex amplification reaction includes at least two primers that are specific for two or more equally abundant target nucleic acid sequences from the plurality of target nucleic acid sequences.

In certain embodiments, a plurality of target nucleic acid sequences with varied abundance are present in a multiplex amplification reaction, and the multiplex amplification reaction includes at least two sets of primers that are specific for two or more similarly abundant target nucleic acid sequences from the plurality of target nucleic acid sequences. In certain embodiments, a plurality of target nucleic acid sequences with varied abundance are present in a multiplex amplification reaction, and the multiplex amplification reaction includes at least two sets of primers that are specific for two or more equally abundant target nucleic acid sequences from the plurality of target nucleic acid sequences.

In certain embodiments, a plurality of target nucleic acid sequences with varied abundance are present in a multiplex amplification reaction, and the multiplex amplification reaction includes at least two primers that are specific for at least a first target nucleic acid sequence and at least a second target nucleic acid sequence from the plurality of target nucleic acid sequences, and the first target nucleic acid sequence is between two and ten-fold more abundant than the second target nucleic acid sequence. In certain embodiments, a plurality of target nucleic acid sequences with varied abundance are present in a multiplex amplification reaction, and the multiplex amplification reaction includes at least two primers that are specific for at least a first target nucleic acid sequence and at least a second target nucleic acid sequence from the plurality of target nucleic acid sequences, and the first target nucleic acid sequence is 10 to 100-fold more abundant than the second target nucleic acid sequence. In certain embodiments, a plurality of target nucleic acid sequences with varied abundance are present in a multiplex amplification reaction, and the multiplex amplification reaction includes at least two primers that are specific for at least a first target nucleic acid sequence and at least a second target nucleic acid sequence from the plurality of target nucleic acid sequences, and the first target nucleic acid sequence is 100 to 1000-fold more abundant than the second target nucleic acid sequence.

In certain embodiments, two or more different multiplex amplification reactions are performed, wherein each different multiplex amplification reaction includes a plurality of target nucleic acid sequences with varied abundance, and at least one multiplex amplification reaction includes at least two primers that are specific for two or more similarly abundant target nucleic acid sequences from the plurality of target nucleic acid sequences, while at least one other multiplex amplification reaction does not include at least two primers that are specific for two or more similarly abundant target nucleic acid sequences from the plurality of target nucleic acid sequences. In certain embodiments, two or more different multiplex amplification reactions are performed, wherein each different multiplex amplification reaction includes a plurality of target nucleic acid sequences with varied abundance, and two or more different multiplex amplification reactions include at least two primers that are specific for two or more similarly abundant target nucleic acid sequences from the plurality of target nucleic acid sequences. In certain embodiments, two or more different multiplex amplification reactions are performed, wherein each different multiplex amplification reaction includes a plurality of target nucleic acid sequences with varied abundance, and each different multiplex amplification reaction includes at least two primers that are specific for two or more similarly abundant target nucleic acid sequences from the plurality of target nucleic acid sequences.

In certain embodiments, two or more different multiplex amplification reactions are performed, wherein each different multiplex amplification reaction includes a plurality of target nucleic acid sequences with varied abundance, at least two probes that are specific for two or more different similarly abundant target nucleic acid sequences, and at least one multiplex amplification reaction includes at least two primers that are specific for two or more similarly abundant target nucleic acid sequences from the plurality of target nucleic acid sequences, while at least one other multiplex amplification reaction does not include at least two primers that are specific for two or more similarly abundant target nucleic acid sequences from the plurality of target nucleic acid sequences. In certain embodiments, two or more different multiplex amplification reactions are performed, wherein each different multiplex amplification reaction includes a plurality of target nucleic acid sequences with varied abundance, at least two probes that are specific for two or more different similarly abundant target nucleic acid sequences, and two or more different multiplex amplification reactions include at least two primers that are specific for two or more similarly abundant target nucleic acid sequences from the plurality of target nucleic acid sequences. In certain embodiments, two or more different multiplex amplification reactions are performed, wherein each different multiplex amplification reaction includes a plurality of target nucleic acid sequences with varied abundance, at least two probes that are specific for two or more different similarly abundant target nucleic acid sequences, and each different multiplex amplification reaction includes at least two primers that are specific for two or more similarly abundant target nucleic acid sequences from the plurality of target nucleic acid sequences.

In certain embodiments, two or more different multiplex amplification reactions are performed, and a plurality of the different multiplex amplification reactions include different primers than the other different multiplex amplification reactions. In certain embodiments, two or more different multiplex amplification reactions are performed, and each of the two or more different multiplex amplification reactions includes at least one different primer set from the other different multiplex amplification reactions. In certain such embodiments, a plurality of the two or more different multiplex amplification reactions amplify two or more target nucleic acid sequences having an abundance that differs from the abundance of the target nucleic acid sequences of at least one other of the two or more different multiplex amplification reactions. In certain such embodiments, a plurality of the two or more different multiplex amplification reactions amplify two or more target nucleic acid sequences having an abundance that differs from the abundance of the target nucleic acid sequences of a plurality of the other different multiplex amplification reactions. For example and not limitation, 96 different multiplex amplification reactions can be performed, with 96 different primer sets, that amplify target nucleic acid sequences having 96 different abundances.

In certain embodiments, most of the genes in a genome are analyzed by performing more than one multiplex amplification reaction. In certain embodiments, most of the genes in a genome are analyzed by performing two or more different multiplex amplification reactions, wherein each different multiplex amplification reaction includes a plurality of target nucleic acid sequences with varied abundance, and at least one multiplex amplification reaction includes at least two primers that are specific for two or more similarly abundant target nucleic acid sequences from the plurality of target nucleic acid sequences, while at least one other multiplex amplification reaction does not include at least two primers that are specific for two or more similarly abundant target nucleic acid sequences form the plurality of target nucleic acid sequences. In certain embodiments, most of the genes in a genome are analyzed by performing two or more different multiplex amplification reactions, wherein each different multiplex amplification reaction includes a plurality of target nucleic acid sequences with varied abundance, and two or more different multiplex amplification reactions include at least two primers that are specific for two or more similarly abundant target nucleic acid sequences from the plurality of target nucleic acid sequences. In certain embodiments, most of the genes in a genome are analyzed by performing two or more different multiplex amplification reactions, wherein each different multiplex amplification reaction includes a plurality of target nucleic acid sequences with varied abundance, and each different multiplex amplification reaction includes at least two primers that are specific for two or more similarly abundant target nucleic acid sequences from the plurality of target nucleic acid sequences.

In certain embodiments, most of the genes in a genome are analyzed by performing two or more different multiplex amplification reactions, and a plurality of the different multiplex amplification reactions include different primers than the other different multiplex amplification reactions. In certain embodiments, most of the genes in a genome are analyzed by performing two or more different multiplex amplification reactions, and each of the two or more different multiplex amplification reactions includes at least one different primer set from the other different multiplex amplification reactions. In certain such embodiments, a plurality of the two or more different multiplex amplification reactions amplify two or more target nucleic acid sequences having an abundance that differs from the abundance of the target nucleic acid sequences of at least one other of the two or more different multiplex amplification reactions. In certain such embodiments, a plurality of the two or more different multiplex amplification reactions amplify two or more target nucleic acid sequences having an abundance that differs from the abundance of the target nucleic acid sequences of a plurality of the other different multiplex amplification reactions. For example and not limitation, a sufficient number of different multiplex amplification reactions can be performed such that the totality of the target nucleic acids in all of the different multiplex amplification reactions represents most of the target nucleic acids in a genome, where each different multiplex amplification reaction includes a different primer set from the other reactions, and each different multiplex amplification reaction amplifies target nucleic acid sequences having different abundances from the target nucleic acid sequences in the other different multiplex amplification reactions.

In certain embodiments, all of the nucleic acids in a transcriptome are analyzed by performing more than one multiplex amplification reaction. In certain embodiments, all of the nucleic acids in a transcriptome are analyzed by performing two or more different multiplex amplification reactions, wherein each different multiplex amplification reaction includes a plurality of target nucleic acid sequences with varied abundance, and at least one multiplex amplification reaction includes at least two primers that are specific for two or more similarly abundant target nucleic acid sequences from the plurality of target nucleic acid sequences, while at least one other multiplex amplification reaction does not include at least two primers that are specific for two or more similarly abundant target nucleic acid sequences form the plurality of target nucleic acid sequences. In certain embodiments, all of the nucleic acids in a transcriptome are analyzed by performing two or more different multiplex amplification reactions, wherein each different multiplex amplification reaction includes a plurality of target nucleic acid sequences with varied abundance, and two or more different multiplex amplification reactions include at least two primers that are specific for two or more similarly abundant target nucleic acid sequences from the plurality of target nucleic acid sequences. In certain embodiments, all of the nucleic acids in a transcriptome are analyzed by performing two or more different multiplex amplification reactions, wherein each different multiplex amplification reaction includes a plurality of target nucleic acid sequences with varied abundance, and each different multiplex amplification reaction includes at least two primers that are specific for two or more similarly abundant target nucleic acid sequences from the plurality of target nucleic acid sequences.

In certain embodiments, all of the nucleic acids in a transcriptome are analyzed by performing two or more different multiplex amplification reactions, and a plurality of the different multiplex amplification reactions include different primers than the other different multiplex amplification reactions. In certain embodiments, all of the nucleic acids in a transcriptome are analyzed by performing two or more different multiplex amplification reactions, and each of the two or more different multiplex amplification reactions includes at least one different primer set from the other different multiplex amplification reactions. In certain such embodiments, a plurality of the two or more different multiplex amplification reactions amplify two or more target nucleic acid sequences having an abundance that differs from the abundance of the target nucleic acid sequences of at least one other of the two or more different multiplex amplification reactions. In certain such embodiments, a plurality of the two or more different multiplex amplification reactions amplify two or more target nucleic acid sequences having an abundance that differs from the abundance of the target nucleic acid sequences of a plurality of the other different multiplex amplification reactions. For example and not limitation, a sufficient number of different multiplex amplification reactions can be performed such that the totality of the target nucleic acids in all of the different multiplex amplification reactions represents all of the nucleic acids in a transcriptome, where each different multiplex amplification reaction includes a different primer set from the other reactions, and each different multiplex amplification reaction amplifies target nucleic acid sequences having different abundances from the target nucleic acid sequences in the other different multiplex amplification reactions.

In certain embodiments, most of the nucleic acids in a transcriptome are analyzed by performing more than one multiplex amplification reaction. In certain embodiments, most of the nucleic acids in a transcriptome are analyzed by performing two or more different multiplex amplification reactions, wherein each different multiplex amplification reaction includes a plurality of target nucleic acid sequences with varied abundance, and at least one multiplex amplification reaction includes at least two primers that are specific for two or more similarly abundant target nucleic acid sequences from the plurality of target nucleic acid sequences, while at least one other multiplex amplification reaction does not include at least two primers that are specific for two or more similarly abundant target nucleic acid sequences form the plurality of target nucleic acid sequences. In certain embodiments, most of the nucleic acids in a transcriptome are analyzed by performing two or more different multiplex amplification reactions, wherein each different multiplex amplification reaction includes a plurality of target nucleic acid sequences with varied abundance, and two or more different multiplex amplification reactions include at least two primers that are specific for two or more similarly abundant target nucleic acid sequences from the plurality of target nucleic acid sequences. In certain embodiments, most of the nucleic acids in a transcriptome are analyzed by performing two or more different multiplex amplification reactions, wherein each different multiplex amplification reaction includes a plurality of target nucleic acid sequences with varied abundance, and each different multiplex amplification reaction includes at least two primers that are specific for two or more similarly abundant target nucleic acid sequences from the plurality of target nucleic acid sequences.

In certain embodiments, most of the nucleic acids in a transcriptome are analyzed by performing two or more different multiplex amplification reactions, and a plurality of the different multiplex amplification reactions include different primers than the other different multiplex amplification reactions. In certain embodiments, most of the nucleic acids in a transcriptome are analyzed by performing two or more different multiplex amplification reactions, and each of the two or more different multiplex amplification reactions includes at least one different primer set from the other different multiplex amplification reactions. In certain such embodiments, a plurality of the two or more different multiplex amplification reactions amplify two or more target nucleic acid sequences having an abundance that differs from the abundance of the target nucleic acid sequences of at least one other of the two or more different multiplex amplification reactions. In certain such embodiments, a plurality of the two or more different multiplex amplification reactions amplify two or more target nucleic acid sequences having an abundance that differs from the abundance of the target nucleic acid sequences of a plurality of the other different multiplex amplification reactions. For example and not limitation, a sufficient number of different multiplex amplification reactions can be performed such that the totality of the target nucleic acids in all of the different multiplex amplification reactions represents most of the nucleic acids in a transcriptome, where each different multiplex amplification reaction includes a different primer set from the other reactions, and each different multiplex amplification reaction amplifies target nucleic acid sequences having different abundances from the target nucleic acid sequences in the other different multiplex amplification reactions.

In certain embodiments, all of the genes in a genome are analyzed by performing more than one multiplex amplification reaction. In certain embodiments, all of the genes in a genome are analyzed by performing two or more different multiplex amplification reactions, wherein each different multiplex amplification reaction includes a plurality of target nucleic acid sequences with varied abundance, and at least one multiplex amplification reaction includes at least two primers that are specific for two or more similarly abundant target nucleic acid sequences from the plurality of target nucleic acid sequences, while at least one other multiplex amplification reaction does not include at least two primers that are specific for two or more similarly abundant target nucleic acid sequences form the plurality of target nucleic acid sequences. In certain embodiments, all of the genes in a genome are analyzed by performing two or more different multiplex amplification reactions, wherein each different multiplex amplification reaction includes a plurality of target nucleic acid sequences with varied abundance, and two or more different multiplex amplification reactions include at least two primers that are specific for two or more similarly abundant target nucleic acid sequences from the plurality of target nucleic acid sequences. In certain embodiments, all of the genes in a genome are analyzed by performing two or more different multiplex amplification reactions, wherein each different multiplex amplification reaction includes a plurality of target nucleic acid sequences with varied abundance, and each different multiplex amplification reaction includes at least two primers that are specific for two or more similarly abundant target nucleic acid sequences from the plurality of target nucleic acid sequences.

In certain embodiments, all of the genes in a genome are analyzed by performing two or more different multiplex amplification reactions, and a plurality of the different multiplex amplification reactions include different primers than the other different multiplex amplification reactions. In certain embodiments, all of the genes in a genome are analyzed by performing two or more different multiplex amplification reactions, and each of the two or more different multiplex amplification reactions includes at least one different primer set from the other different multiplex amplification reactions. In certain such embodiments, a plurality of the two or more different multiplex amplification reactions amplify two or more target nucleic acid sequences having an abundance that differs from the abundance of the target nucleic acid sequences of at least one other of the two or more different multiplex amplification reactions. In certain such embodiments, a plurality of the two or more different multiplex amplification reactions amplify two or more target nucleic acid sequences having an abundance that differs from the abundance of the target nucleic acid sequences of a plurality of the other different multiplex amplification reactions. For example and not limitation, a sufficient number of different multiplex amplification reactions can be performed such that the totality of the target nucleic acids in all of the different multiplex amplification reactions represents all of the genes in a genome, where each different multiplex amplification reaction includes a different primer set from the other reactions, and each different multiplex amplification reaction amplifies target nucleic acid sequences having different abundances from the target nucleic acid sequences in the other different multiplex amplification reactions.

In certain embodiments, the abundance of certain target nucleic acid sequences is determined experimentally for a particular sample containing the target nucleic acid sequences (see, e.g., published U.S. Patent Application No. 2004/0121371 A1). In certain embodiments, the abundance of one or more target nucleic acid sequences in a particular sample is determined by gene expression analysis. In certain embodiments, the relative abundance of two or more target nucleic acid sequences in a particular sample is determined by gene expression analysis. In certain embodiments, the gene expression analysis is performed using real-time PCR. In certain embodiments, the gene expression analysis is performed using a hybridization-based microarray (e.g., the Applied Biosystems 1700 Microarray Analyzer or Affymetrix GeneChip® systems. In certain embodiments, the gene expression analysis is performed by other gene expression measurement technologies known in the art. In certain embodiments, an experimental determination of the abundance of certain target nucleic acid sequences for a particular sample is used to predict the abundance of those target nucleic acid sequences in one or more similar samples.

In certain embodiments, the expression of individual target nucleic acid sequences from a single source is determined using one or more of the above-described gene expression methods. In certain embodiments, the expression of individual target nucleic acid sequences from different sources is determined using one or more of the above-described gene expression methods.

In certain embodiments, the C_{T} for a target nucleic acid sequence in an amplification reaction is indicative of its abundance in the amplification reaction. In certain embodiments, a panel of target nucleic acid sequences is analyzed in a series of multiplex amplification reactions in which each target nucleic acid sequence is co-amplified with the same reference nucleic acid sequence. In certain such embodiments, the C_{T} for each target nucleic acid sequence in each reaction is determined. In certain such embodiments, the C_{T} for the reference nucleic acid sequence in each reaction is also determined. In certain such embodiments, the C_{T} for each target nucleic acid sequence from each reaction is normalized using the C_{T} for the reference nucleic acid sequence in each reaction such that the C_{T} for each target nucleic acid sequence in different reactions may be fairly compared. In certain embodiments, a panel of target nucleic acid sequences is analyzed in a series of multiplex amplification reactions in which each target nucleic acid sequence is co-amplified with the same reference nucleic acid sequence, the C_{T} for each target nucleic acid sequence in the panel is determined and subsequently normalized to the C_{T} for the reference nucleic acid sequence, and the results are included in a database of the normalized C_{T} values for each target nucleic acid sequence in the panel. In certain embodiments, a panel of target nucleic acid sequences is analyzed in a series of multiplex amplification reactions including one or more amplification reactions of a reference nucleic acid sequence, wherein the C_{T} for each target nucleic acid sequence in the panel is determined and subsequently normalized to the C_{T} for the reference nucleic acid sequence, and the results are included in a database of the normalized C_{T} values for each target nucleic acid sequence in the panel. In certain embodiments, the reference nucleic acid sequence is included at least two multiplex amplification reactions. In certain embodiments, the reference nucleic acid sequence is not included in the one or more multiplex amplification reactions, but is amplified simultaneously under the same conditions as the one or more multiplex amplification reactions.

In certain embodiments, the distribution of the types of tissue in which a target nucleic acid sequences is expressed is indicative of its abundance. In certain embodiments, very abundant target nucleic acid sequences may be at least somewhat expressed in a variety of different types of tissues. In certain embodiments, target nucleic acid sequences that are low to moderately abundant may be expressed in one or a few different types of tissues.

In certain embodiments, two or more wells of a multiwell plate each contain two or more primers specific for two or more target nucleic acid sequences of similar abundance in a particular sample, wherein the abundance of the target nucleic acid sequences was determined by gene expression data as described above. In certain embodiments, two or more wells of a multiwell plate each contain two or more primers specific for two or more target nucleic acid sequences of equal abundance in a particular sample, wherein the abundance of the target nucleic acid sequences was determined by gene expression data as described above.

In certain embodiments, pools of primer sets selected to amplify specific target nucleic acid sequences of similar abundance are designed. In certain embodiments, one or more pools of primer sets selected to amplify specific target nucleic acid sequences of similar abundance are designed by combining primer sets that amplify target nucleic acid sequences having similar C_{T}. In certain embodiments, one or more pools of primer sets selected to amplify specific target nucleic acid sequences of similar abundance are designed by combining primer sets that amplify target nucleic acid sequences having equal C_{T}. In certain embodiments, one or more pools of primer sets selected to amplify specific target nucleic acid sequences of similar abundance are designed by combining primer sets that amplify target nucleic acid sequences having similar expression in a variety of different types of tissues. In certain embodiments, one or more pools of primer sets selected to amplify specific target nucleic acid sequences of equal abundance are designed by combining primer sets that amplify target nucleic acid sequences having equal expression in a variety of different types of tissues.

In certain embodiments, abundance data from a single source is used to design one or more pools of primer sets. In certain embodiments, abundance data from several samples of diverse origin is used to design one or more pools of primer sets.

In certain embodiments, an optimized reagent mixture is included in a multiplex amplification reaction to reduce amplification bias. In certain embodiments, a plurality of target nucleic acid sequences with varied abundance are present in a multiplex amplification reaction, the multiplex amplification reaction includes at least two primers that are specific for two or more similarly abundant target nucleic acid sequences from the plurality of target nucleic acid sequences, and the reaction further includes an optimized reagent mixture. In certain embodiments, a plurality of target nucleic acid sequences with varied abundance are present in a multiplex amplification reaction, the multiplex amplification reaction includes at least two primers that are specific for two or more equally abundant target nucleic acid sequences from the plurality of target nucleic acid sequences, and the reaction further includes an optimized reagent mixture. In certain embodiments, a plurality of target nucleic acid sequences with varied abundance are present in a multiplex amplification reaction, and the multiplex amplification reaction includes at least two primer sets that are specific for two or more similarly abundant target nucleic acid sequences from the plurality of target nucleic acid sequences, and the reaction further includes an optimized reagent mixture. In certain embodiments, a plurality of target nucleic acid sequences with varied abundance are present in a multiplex amplification reaction, and the multiplex amplification reaction includes at least two primer sets that are specific for two or more equally abundant target nucleic acid sequences from the plurality of target nucleic acid sequences, and the reaction further includes an optimized reagent mixture.

An “optimized reagent mixture” is a mixture of reagents used in an amplification reaction that has been modified so as to minimize any amplification bias. In certain embodiments, an optimized reagent mixture includes one or more reagents in an amount increased from the amounts typically found in the art (e.g., as described in (Sambrook and Russell, Molecular Cloning: A Laboratory Manual, 3^{rd} ed. (2001), Chapter 8: In Vitro Amplification of DNA by the Polymerase Chain Reaction, page 21). In certain embodiments, the amount of polymerase in the optimized reagent mixture is increased from the 0.01 to 0.05 U/μL typically used in the art. In certain embodiments, the amount of polymerase in the optimized reagent mixture is increased from two to ten-fold from the 0.01 to 0.05 U/μL typically used in the art. In certain embodiments, an additional 0.05, 0.1, 0.15, 0.2, 0.25, 0.3, 0.35, 0.4, 0.45, or 0.5 U/μL of polymerase is included in the optimized reagent mix in addition to the 0.01 to 0.05 U/μL typically used in the art. In certain embodiments, the amount of dNTPs in the optimized reagent mixture is increased from the 200 μM typically used in the art. In certain embodiments, the amount of dNTPs in the optimized reagent mixture is increased from two to ten-fold from the 200 μM to 1 mM typically used in the art. In certain embodiments, an additional 0.5, 1.0, 1.5, 2.0, 2.5, 3.0, 3.5, 4.0, 4.5, or 5.0 mM dNTPs is included in the optimized reagent mix in addition to the 200 μM to 1 mM dNTPs typically used in the art. In certain embodiments, the amount of magnesium ions in the optimized reagent mixture is increased from the 1.5 mM typically used in the art. In certain embodiments, the amount of magnesium ions in the optimized reagent mixture is increased from two to ten-fold from the 1.5 mM typically used in the art. In certain embodiments, an additional 0.25, 0.5, 0.75, 1.0, 1.25, 1.5, 1.75, or 2.0 mM magnesium ions is included in the optimized reagent mix in addition to the 1.5 mM magnesium ions typically used in the art. In certain embodiments, at least two of the amount of polymerase, the amount of dNTPs, and the amount of magnesium ions in the optimized reagent mixture are increased from the amount(s) typically used in the art.

In certain embodiments, an optimized reagent mixture is based on TaqMan® Universal PCR Master Mix (Applied Biosystems, Product No. 4304437). In certain embodiments, 0.20 U/μL TaqGold enzyme is added to the TaqMan® Universal PCR Master Mix. In certain embodiments, 0.25 U/μL TaqGold enzyme is added to the TaqMan® Universal PCR Master Mix. In certain embodiments, 2 mM dNTP is added to the TaqMan® Universal PCR Master Mix. In certain embodiments, 1 mM magnesium ions is added to the TaqMan® Universal PCR Master Mix. In certain embodiments, 1 mM magnesium ions, 2 mM dNTPs, and 0.20 U/μL TaqGold enzyme is added to the TaqMan® Universal PCR Master Mix.

In certain embodiments, at least one of the primers in a multiplex amplification reaction is at an optimized primer concentration to reduce amplification bias. In certain embodiments, a multiplex amplification reaction includes a plurality of target nucleic acid sequences with varied abundance and at least two primers that are specific for two or more similarly abundant target nucleic acid sequences from the plurality of target nucleic acid sequences, wherein at least one of the primers in the multiplex amplification reaction is at an optimized primer concentration (see, e.g., Applied Biosystems User Bulletin #5 for ABI Prism 7700 Sequence Detection System, “Multiplex PCR with TaqMan VIC Probes”). In certain embodiments, a multiplex amplification reaction includes a plurality of target nucleic acid sequences with varied abundance and at least two primer sets that are specific for two or more similarly abundant target nucleic acid sequences from the plurality of target nucleic acid sequences, wherein at least one of the primers in at least two primer sets in the multiplex amplification reaction is at an optimized primer concentration. An “optimized primer concentration” refers to a primer concentration that is modified so as to minimize amplification bias. In certain embodiments, a lowered concentration of a primer specific for a more-abundant target nucleic acid sequence is an optimized primer concentration. In certain embodiments, an increased concentration of a primer specific for a less-abundant target nucleic acid sequence is an optimized primer concentration. In certain embodiments, a lowered concentration of a primer specific for a more-abundant target nucleic acid sequence and an increased concentration of a primer specific for a less-abundant target nucleic acid sequence is an optimized primer concentration. In certain embodiments, one or more primer concentrations are modified such that the amplification of one target nucleic acid sequence in a multiplex amplification reaction is limited by the concentration of one or more primers in the reaction. In certain embodiments, one or more primer concentrations are modified such that the amplifications of two or more target nucleic acid sequences in a multiplex amplification reaction are limited by the concentration of one or more primers in the reaction.

In certain embodiments, the extension time of the amplification reaction is modified such that amplification bias is minimized. In certain embodiments, a multiplex amplification reaction includes a plurality of target nucleic acid sequences with varied abundance, and also includes at least two primers that are specific for two or more similarly abundant target nucleic acid sequences from the plurality of target nucleic acid sequences, and the extension time of the amplification reaction is modified such that amplification bias is minimized. The “extension time” is the time during which a primer extension reaction takes place. In certain embodiments, amplification bias is minimized by increasing the extension time of the amplification reaction. In certain embodiments, the extension time is increased from the typical extension time of 15 to 30 seconds to between 30 seconds and 60 seconds. In certain embodiments, the extension time is increased from the typical extension time of 15 to 30 seconds to between 60 seconds and 90 seconds. In certain embodiments, the extension time is increased from the typical extension time of 15 to 30 seconds to between 90 seconds and 120 seconds. In certain embodiments, the extension time is increased from the typical extension time of 15 to 30 seconds to between two minutes and five minutes.

In certain embodiments, the extension temperature is modified such that amplification bias is minimized. In certain embodiments, a multiplex amplification reaction includes a plurality of target nucleic acid sequences with varied abundance, and also includes at least two primers that are specific for two or more similarly abundant target nucleic acid sequences from the plurality of target nucleic acid sequences, and the extension temperature of the amplification reaction is modified such that amplification bias is minimized. The “extension temperature” is the temperature at which a primer extension reaction takes place. In certain embodiments, the extension temperature is set as the temperature at which the extension rate of a polymerase in the amplification reaction is maximized. In certain embodiments, amplification bias is minimized by increasing the extension temperature of the amplification reaction. In certain embodiments, amplification bias is minimized by decreasing the extension temperature of the amplification reaction. In certain embodiments, the extension temperature is increased by 2 to 5 degrees from the temperature at which the extension rate of the polymerase is optimized. In certain embodiments, the extension temperature is increased by 5 to 10 degrees from the temperature at which the extension rate of the polymerase is optimized. In certain embodiments, the extension temperature is increased by 10 to 15 degrees from the temperature at which the extension rate of the polymerase is optimized. In certain embodiments, the extension temperature is increased by 15 to 20 degrees from the temperature at which the extension rate of the polymerase is optimized. In certain embodiments, the melting temperature of one or more primers specific for one or more less-abundant target nucleic acid sequences in an amplification reaction can be designed to be higher than the melting temperature of one or more primers specific for one or more more-abundant target nucleic acid sequences. In certain embodiments, the melting temperature of one or more primers specific for one or more more-abundant target nucleic acid sequences in an amplification reaction can be designed to be higher than the melting temperature of one or more primers specific for one or more less-abundant target nucleic acid sequences.

In certain embodiments, preamplification of one or more target nucleic acid sequences is performed prior to inclusion of the one or more target nucleic acid sequences in a multiplex amplification reaction (see, e.g., published U.S. Patent Application No. 2004-0014105 A1). In certain such embodiments, a sample may be selectively enriched for one or more target nucleic acid sequences by subjecting the sample to one or more rounds of amplification in which amplification of certain target nucleic acid sequences is blocked. In certain such embodiments, one or more enzymatically non-extendable nucleobase oligomers specific for one or more moderate-to-high abundance target nucleic acid sequence is included in the preamplification reaction. In certain such embodiments, the one or more non-extendable nucleobase oligomers bind to the one or more moderate-to-high abundance target nucleic acid sequences and prevent amplification of those sequences, while amplification of other target nucleic acid sequences in the reaction proceeds unimpeded. In certain such embodiments, the amplified products of the preamplification reaction are used as target nucleic acid sequences in one or more multiplex amplification reactions.

In certain embodiments, a plurality of target nucleic acid sequences with varied abundance are present in a multiplex amplification reaction, and the multiplex amplification reaction includes at least two sets of primers that are specific for two or more similarly abundant target nucleic acid sequences from the plurality of target nucleic acid sequences, and also includes at least one enzymatically non-extendable nucleobase oligomer (see, e.g., published U.S. Patent Application No. 2004-0014105 A1) specific for one or more target nucleic acid sequence which is not desired to be amplified. In certain such embodiments, at least one enzymatically non-extendable nucleobase oligomer binds to one or more of the plurality of target nucleic acid sequences and prevents amplification of the target nucleic acid sequences to which the enzymatically non-extendable nucleobase oligomer is bound. In certain such embodiments, the one or more target nucleic acid sequences to which at least one enzymatically non-extendable nucleobase oligomer binds is in similar abundance in the reaction to at least one other target nucleic acid sequence that is amplified in the reaction. In certain such embodiments, the one or more target nucleic acid sequences to which at least one enzymatically non-extendable nucleobase oligomer binds is not in similar abundance in the reaction to at least one other target nucleic acid sequence that is amplified in the reaction.

In certain embodiments, a first target nucleic acid sequence is present in similar abundance to a second target nucleic acid sequence in a multiplex amplification reaction. In certain embodiments, a first target nucleic acid sequence is present in equal abundance to a second target nucleic acid sequence in a multiplex amplification reaction. In certain embodiments, a first target nucleic acid sequence is five to ten-fold more abundant than a second target nucleic acid sequence in a multiplex amplification reaction. In certain embodiments, a first target nucleic acid sequence is 10 to 100-fold more abundant than a second target nucleic acid sequence in a multiplex amplification reaction. In certain embodiments, a first target nucleic acid sequence is 100 to 1000-fold more abundant than a second target nucleic acid sequence in a multiplex amplification reaction.

In certain embodiments, two or more target nucleic acid sequences in a multiplex amplification reaction are present in similar abundance. In certain embodiments, two or more target nucleic acid sequences in a multiplex amplification reaction are present in equal abundance.

In certain embodiments, two or more different multiplex amplification reactions are performed. In certain such embodiments, at least one multiplex amplification reaction includes two or more target nucleic acid sequences having similar abundance while at least one other multiplex amplification reaction includes two or more target nucleic acid sequences not having similar abundance. In certain such embodiments, two or more multiplex amplification reactions each include two or more target nucleic acid sequences having similar abundance. In certain such embodiments, a plurality of multiplex amplification reactions each include two or more target nucleic acid sequences having similar abundance.

In certain embodiments, most of the genes of a genome are analyzed by performing more than one multiplex amplification reaction, wherein one of the multiplex amplification reactions may include two or more target nucleic acid sequences having similar abundance while another multiplex amplification reaction may include two or more target nucleic acid sequences not having similar abundance. In certain embodiments, most of the genes of a genome are analyzed by performing more than one multiplex amplification reaction, wherein two or more of the multiplex amplification reactions may each include two or more target nucleic acid sequences having similar abundance. In certain embodiments, most of the genes of a genome are analyzed by performing more than one multiplex amplification reaction, wherein a plurality of the multiplex amplification reactions each include two or more target nucleic acid sequences having similar abundance.

In certain embodiments, all of the genes in a genome are analyzed by performing more than one multiplex amplification reaction, wherein one of the multiplex amplification reactions may include two or more target nucleic acid sequences having similar abundance while another multiplex amplification reaction may include two or more target nucleic acid sequences not having similar abundance. In certain embodiments, all of the genes in a genome are analyzed by performing more than one multiplex amplification reaction, wherein two or more of the multiplex amplification reactions may each include two or more target nucleic acid sequences having similar abundance. In certain embodiments, all of the genes in a genome are analyzed by performing more than one multiplex amplification reaction, wherein a plurality of the multiplex amplification reactions each include two or more target nucleic acid sequences having similar abundance.

In certain embodiments, most of the nucleic acids in a transcriptome are analyzed by performing more than one multiplex amplification reaction, wherein one of the multiplex amplification reactions may include two or more target nucleic acid sequences having similar abundance while another multiplex amplification reaction may include two or more target nucleic acid sequences not having similar abundance. In certain embodiments, most of the nucleic acids in a transcriptome are analyzed by performing more than one multiplex amplification reaction, wherein two or more of the multiplex amplification reactions may each include two or more target nucleic acid sequences having similar abundance. In certain embodiments, most of the nucleic acids in a transcriptome are analyzed by performing more than one multiplex amplification reaction, wherein a plurality of the multiplex amplification reactions each include two or more target nucleic acid sequences having similar abundance.

In certain embodiments, all of the nucleic acids in a transcriptome are analyzed by performing more than one multiplex amplification reaction, wherein one of the multiplex amplification reactions may include two or more target nucleic acid sequences having similar abundance while another multiplex amplification reaction may include two or more target nucleic acid sequences not having similar abundance. In certain embodiments, all of the nucleic acids in a transcriptome are analyzed by performing more than one multiplex amplification reaction, wherein two or more of the multiplex amplification reactions may each include two or more target nucleic acid sequences having similar abundance. In certain embodiments, all of the nucleic acids in a transcriptome are analyzed by performing more than one multiplex amplification reaction, wherein a plurality of the multiplex amplification reactions each include two or more target nucleic acid sequences having similar abundance.

In certain embodiments, abundance data from a single source is used to design one or more pools of target nucleic acid sequences suitable for multiplex amplification, based on expected target abundance. In certain embodiments, abundance data from several samples of diverse origin is used to design one or more pools of target nucleic acid sequences suitable for multiplex amplification, based on expected target abundance.

In certain embodiments, one or more experimental pools of target nucleic acid sequences suitable for multiplex amplification are designed such that the target nucleic acid sequences amplified by a multiplex amplification reaction are found in similar abundance to their abundance in a typical sample. In certain embodiments, one or more experimental pools of target nucleic acid sequences suitable for multiplex amplification are designed such that the target nucleic acid sequences amplified by a multiplex amplification reaction measured by the pooled assays are found in equivalent abundance to their abundance in a typical sample. A “typical sample” is a sample that is representative of the samples in which one or more target nucleic acid sequences is found. In certain embodiments, a typical sample may be drawn from different tissues of the same species.

In certain embodiments, a first target nucleic acid sequence is present in similar abundance to a second target nucleic acid sequence in a multiplex amplification reaction, and the reaction further includes an optimized reagent mixture. In certain embodiments, a first target nucleic acid sequence is present in equal abundance to a second target nucleic acid sequence in a multiplex amplification reaction, and the reaction further includes an optimized reagent mixture. In certain embodiments, a first target nucleic acid sequence is between two and ten-fold more abundant than a second target nucleic acid sequence in a multiplex amplification reaction, and the reaction further includes an optimized reagent mixture. In certain embodiments, a first target nucleic acid sequence is 10 to 100-fold more abundant than a second target nucleic acid sequence in a multiplex amplification reaction, and the reaction further includes an optimized reagent mixture. In certain embodiments, a first target nucleic acid sequence is 100 to 1000-fold more abundant than a second target nucleic acid sequence in a multiplex amplification reaction, and the reaction further includes an optimized reagent mixture.

In certain embodiments, a first target nucleic acid sequence is provided in similar abundance to at least a second target nucleic acid sequence in a multiplex amplification reaction, and the reaction further includes an optimized primer concentration for one or more of the primers in the amplification reaction (see, e.g., Applied Biosystems User Bulletin #5 for ABI Prism 7700 Sequence Detection System, “Multiplex PCR with TaqMan VIC Probes”).

In certain embodiments, a first target nucleic acid sequence is provided in similar abundance to at least a second target nucleic acid sequence in a multiplex amplification reaction, and the extension time of the amplification reaction is modified such that amplification bias is minimized. In certain embodiments, a first target nucleic acid sequence is provided in similar abundance to at least a second target nucleic acid sequence in a multiplex amplification reaction, and the extension time is increased from the typical extension time of 15 to 30 seconds to between 30 seconds and 60 seconds, to between 60 seconds and 90 seconds, to between 90 seconds and 120 seconds, or to between two minutes and five minutes. In certain embodiments, a first target nucleic acid sequence is provided in similar abundance to at least a second target nucleic acid sequence in a multiplex amplification reaction, and the extension temperature of the amplification reaction is modified such that amplification bias is minimized. In certain embodiments, a first target nucleic acid sequence is provided in similar abundance to at least a second target nucleic acid sequence in a multiplex amplification reaction, and the extension temperature is increased by 2 to 5 degrees from the temperature at which the extension rate of the polymerase is optimized. In certain embodiments, a first target nucleic acid sequence is provided in similar abundance to at least a second target nucleic acid sequence in a multiplex amplification reaction, and the extension temperature is increased by 5 to 10 degrees from the temperature at which the extension rate of the polymerase is optimized. In certain embodiments, a first target nucleic acid sequence is provided in similar abundance to at least a second target nucleic acid sequence in a multiplex amplification reaction, and the extension temperature is increased by 10 to 15 degrees from the temperature at which the extension rate of the polymerase is optimized. In certain embodiments, a first target nucleic acid sequence is provided in similar abundance to at least a second target nucleic acid sequence in a multiplex amplification reaction, and the extension temperature is increased by 15 to 20 degrees from the temperature at which the extension rate of the polymerase is optimized.

### EXAMPLES

### Example 1

This study investigated the effect of modifying a standard reagent mixture used in multiplex quantitative real-time TaqMan PCR on the amplification of two target nucleic acid sequences with differing abundance in the same reaction.

Two target nucleic acid sequences, portions of interleukin-18 (“IL-18”) (GenBank reference NM_{—}001562) and glyceraldehyde-3-phosphate dehydrogenase (“GAPDH”) (GenBank reference NM_{—}002046), were separately amplified by PCR. The primers used to amplify the target nucleic acid sequence within IL-18 were GGCTGTAACTATCTCTGTGAAGTGTGA (SEQ ID NO: 1) and TCCTGGGACACTTCTCTGAAAGA (SEQ ID NO: 2). The primers used to amplify the target nucleic acid sequence within GAPDH were AGCCGAGCCACATCGCT (SEQ ID NO: 3) and TGGCMCAATATCCACTTTACCAGAGT (SEQ ID NO: 4). The amplification conditions were 10 minutes at 95° C., and then 40 cycles of 15 seconds at 95° C. followed by 60 seconds at 60° C., where each cycle included the 95° C. step and the 60° C. step. The IL-18 and GAPDH amplification products (amplicons) were purified using a Qiaquick column (Qiagen Inc.), according to the manufacturer's instructions. The predicted sequence of the amplicon from the target nucleic acid within IL-18 (the “IL-18 amplicon”) was GGCTGTAACTATCTCTGTGAAGTGTGAGMAATTTCAACTCTCTCCTGTGAGAACA AAATTATTTCCTTTAAGGAAATGAATCCTCCTGATMCATCAAGGATACAMMGTG ACATCATATTCTTTCAGAGMGTGTCCCAGGA (SEQ ID NO: 5). The predicted sequence of the amplicon from the target nucleic acid within GAPDH (the “GAPDH amplicon”) was AGCCGAGCCACATCGCTCAGACACCATGGGGAAGGTGAAGGTCGGAGTCAACGG ATTTGGTCGTATTGGGCGCCTGGTCACCAGGGCTGCTTTTAACTCTGGTAAAGTGG ATATTGTTGCCA (SEQ ID NO: 6). The purified IL-18 and GAPDH amplicons were used as target nucleic acid sequences for further experimentation.

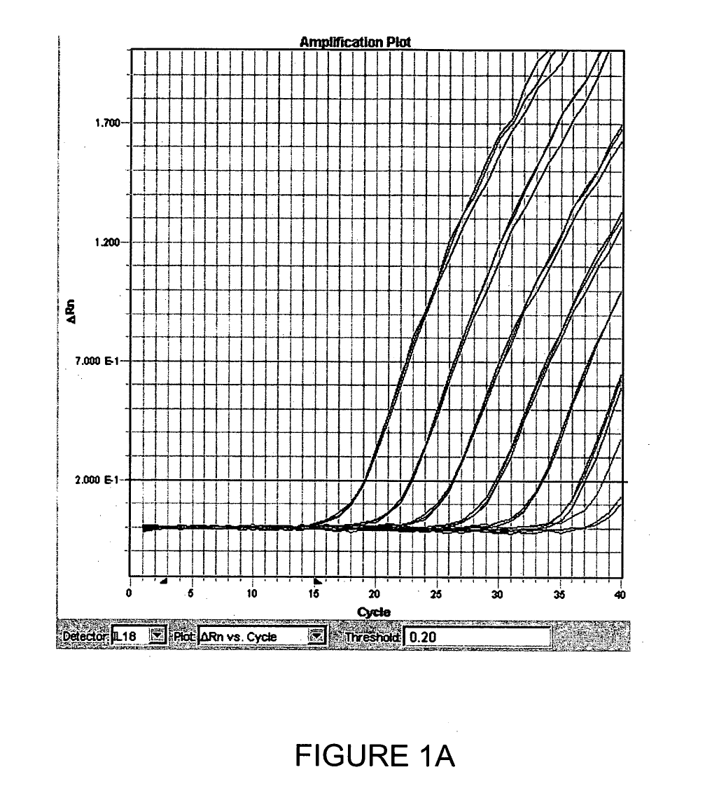

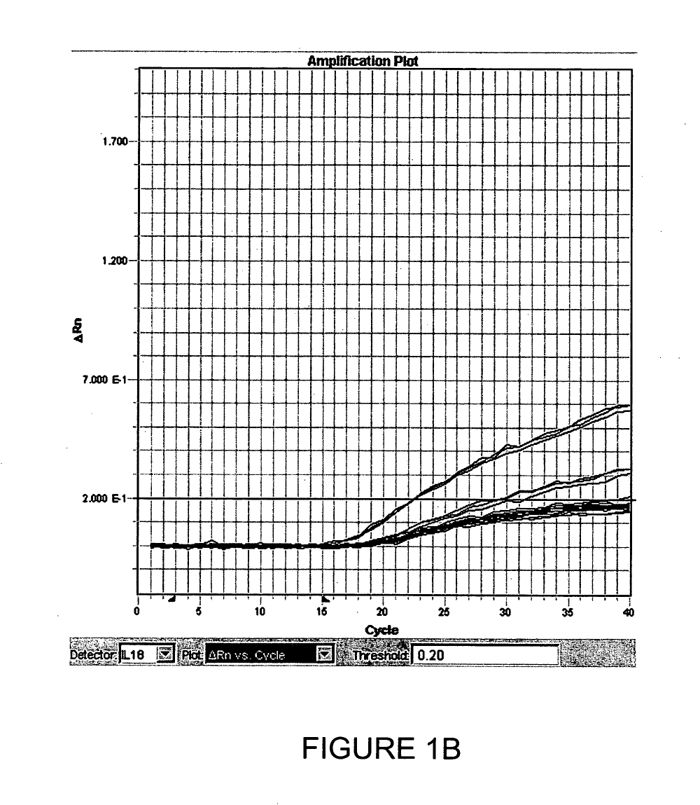

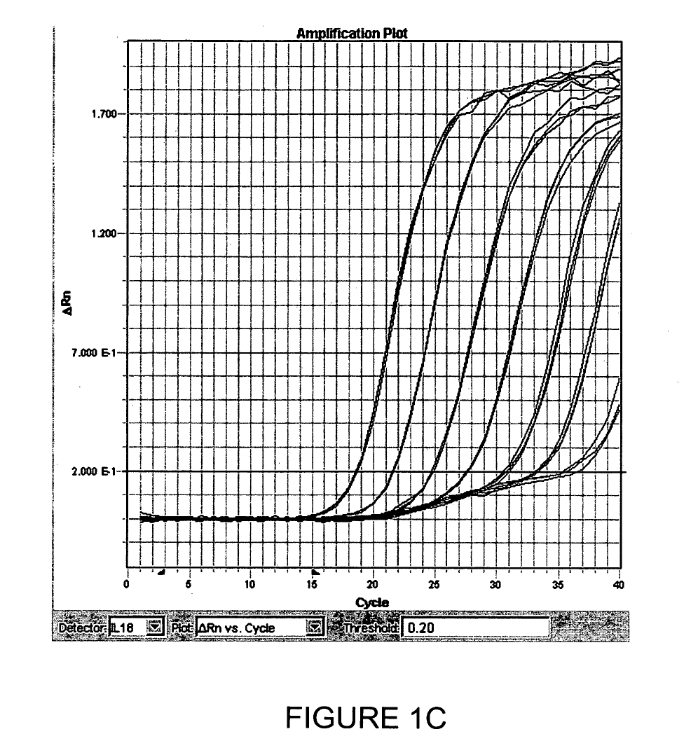

A series of seven control singleplex quantitative real-time PCR reactions was performed. The series of reactions contained a variable amount of purified IL-18 amplicon (varied as a ten-fold serial dilution) and a constant amount of purified GAPDH amplicon (held constant at a level equal, as determined by C_{T}, to the highest concentration of the IL-18 amplicon). Each reaction included 450 nM IL-18-specific primers described in the paragraph above and 125 nM IL-18-specific Taqman probes labeled with the reporter fluorochrome 6-carboxyfluorescein (“FAM”) with the sequence (CCTTTAAGGMATGAATCC (SEQ ID NO: 7)), but no primers or probes for GAPDH. Universal Master Mix (Applied Biosystems) was used in the amplification reactions. Amplification and real-time analysis were performed in an ABI Prism 7900 Sequence Detection System. Amplification cycle parameters were: 10 minutes at 95° C., followed by 40 cycles of 95° C. for 15 seconds and 60° C. for 1 minute. Amplification data was depicted graphically as a plot of cycle number versus the magnitude of the fluorescent signal, normalized by the subtraction of a baseline. The baseline was calculated from the signal from the early cycles of each reaction, before PCR reaction products were detectable (ΔRn). As shown in FIG. 1A, significant amplification of the target nucleic acid sequence within IL-18 was observed in all reactions, and observed C_{T} was increased with decreasing concentration of IL-18 target nucleic acid sequence in the reaction.

A second series of seven different multiplex real-time TaqMan PCR reactions was performed, including both IL-18- and GAPDH-specific primers (each at a concentration of 450 nM) and TaqMan probes (each at a concentration of 125 nM) and the same concentrations of the IL-18 and GADPH amplicons that were used in the singleplex reactions discussed above. The TaqMan probe used to detect GAPDH target nucleic acid sequence was labeled with VIC™ and had the sequence (CCCTGGTGACCAGGC (SEQ ID NO: 8)). Universal Master Mix (Applied Biosystems) was used in the amplification reactions. The amplification reaction parameters were identical to those described above for the first series of reactions. The results are depicted in FIG. 1B. When the GAPDH primers and TaqMan probes were included in the multiplex PCR reactions, amplification of IL-18 amplicon was significantly impaired at all concentrations of the IL-18 amplicon, and particularly at the lower relative target abundance dilutions (compare FIG. 1B with FIG. 1A).

A third series of multiplex real-time TaqMan PCR reactions was performed, containing both IL-18 and GAPDH primers and TaqMan probes, as described above, and the same concentrations of the IL-18 and GAPDH amplicons as described above. Universal Master Mix was replaced by optimized Master Mix. Optimized Master Mix contained the same reagents as Universal Master Mix (dideoxy nucleotide triphosphates, buffer, magnesium chloride, and TaqGold polymerase), but included five times the amount of TaqGold polymerase, or 0.25 U/μL, and three times the amount of dNTPs, or 3 mM. All other conditions remained the same as in the multiplex real-time TaqMan PCR reactions discussed above (see FIG. 1B). The amplification reaction parameters were identical to those described above for the first series of reactions. The results are depicted in FIG. 1C. Replacement of the Universal Master Mix with optimized Master Mix restored amplification of IL-18 amplicon at all concentrations of the IL-18 amplicon to levels comparable to the singleplex levels (compare FIG. 1C to FIGS. 1B and 1A).

### Example 2

As shown in Example 1, when the purified IL-18 amplicon was present in a multiplex PCR reaction at much lower abundance than the purified GAPDH amplicon, amplification of IL-18 amplicon was significantly reduced from amplification of IL-18 amplicon observed in the absence of the GAPDH-specific primers and TaqMan probes (compare FIG. 1B to FIG. 1A). This study further investigated differences in amplification efficiency in multiplex quantitative real-time TaqMan PCR reactions in which the target nucleic acid sequence cDNAs have similar or differing abundance.

Applied Biosystems TaqMan Gene Expression Assays specific for three human genes, Hs00155659 (AHSG), Hs00174099 (IL1RN), and Hs00234981 (SCYA14) were previously performed using cDNA from a Human Universal Reference RNA (Stratagene), and thus all three had known target abundance. (Hs00155659 (AHSG) and Hs00174099 (IL1RN) are commercially available FAM-labeled Assays-On-Demand (Applied Biosystems); Hs00234981 (SCYA14) is a custom-manufactured VIC-labeled assay made using the commercial sequence design). The forward primer for HS00234981 was CGTCAGCGGATTATGGATTACTATG (SEQ ID NO: 9), and the reverse primer for Hs00234981 was ACGGMTGGCCCCTTTTG (SEQ ID NO: 10). The Hs00234981 TaqMan probe used was (VIC)TGATGAAGACAATTCC(MGB) (SEQ ID NO: 11), where “MGB” is the minor groove binding molecule (Applied Biosystems). The concentrations of primers and probes used were identical to those in Example 1. Two sets of multiplex real-time TaqMan PCR reactions, four reactions in each set, were performed, using Universal Master Mix. The amplification reaction parameters were identical to those described in Example 1.

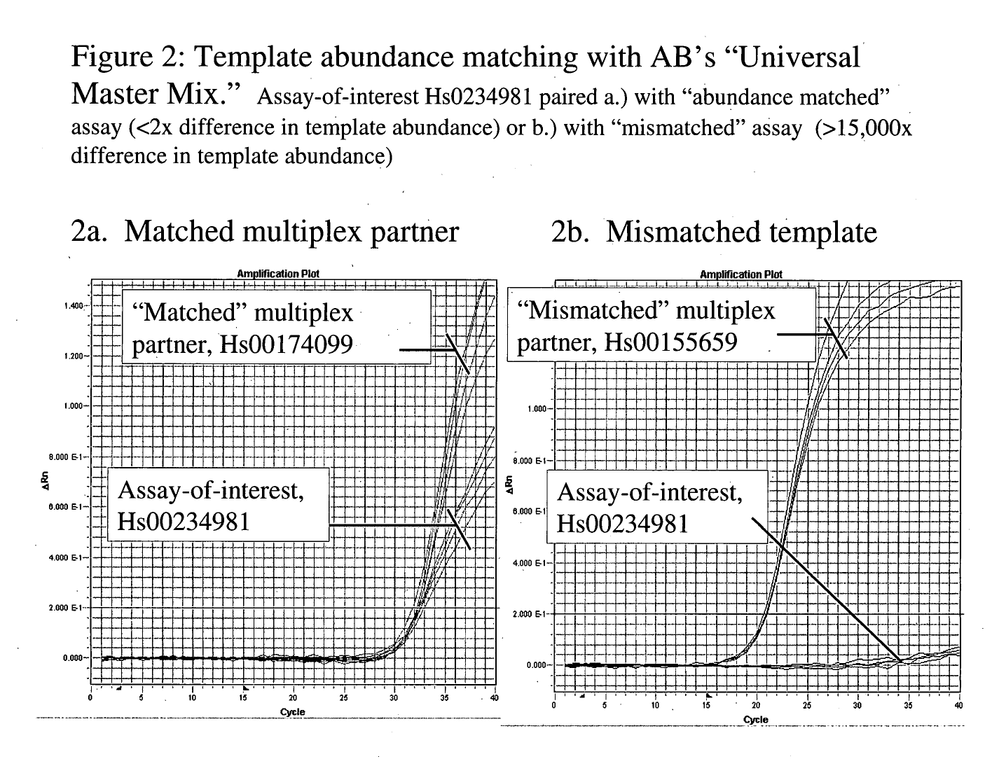

In a first set of reactions (FIG. 2a), two similarly abundant target nucleic acid sequences were multiplex amplified from Human Universal Reference cDNA with the Hs00174099 (FAM) and HS00234981 (VIC) assays. As shown in FIG. 2a, amplification of both target nucleic acid sequences was observed, with a C_{T} of about 32 for both target amplifications. Some variation in ΔRn between different assays was treated as normal, likely due to manufacturing variation or to primer/probe designs, so long as amplification progressed well above the threshold ΔRn value. In a second set of reactions (FIG. 2b), the same Hs00234981 (VIC) assay as above was multiplex amplified with a Hs00155659 (FAM) assay. As shown in FIG. 2b, significant amplification of Hs00155659 was observed, with a measured C_{T} of about 21, while amplification of Hs00234981 was not observed. Thus, using Universal Master Mix, Hs00234981 was amplified in a multiplex PCR reaction when the other target nucleic acid sequence was of similar abundance to Hs00234981, but was not amplified in such a reaction when in the presence of a more abundant (estimated 2000 times more abundant) target nucleic acid sequence.

### Example 3

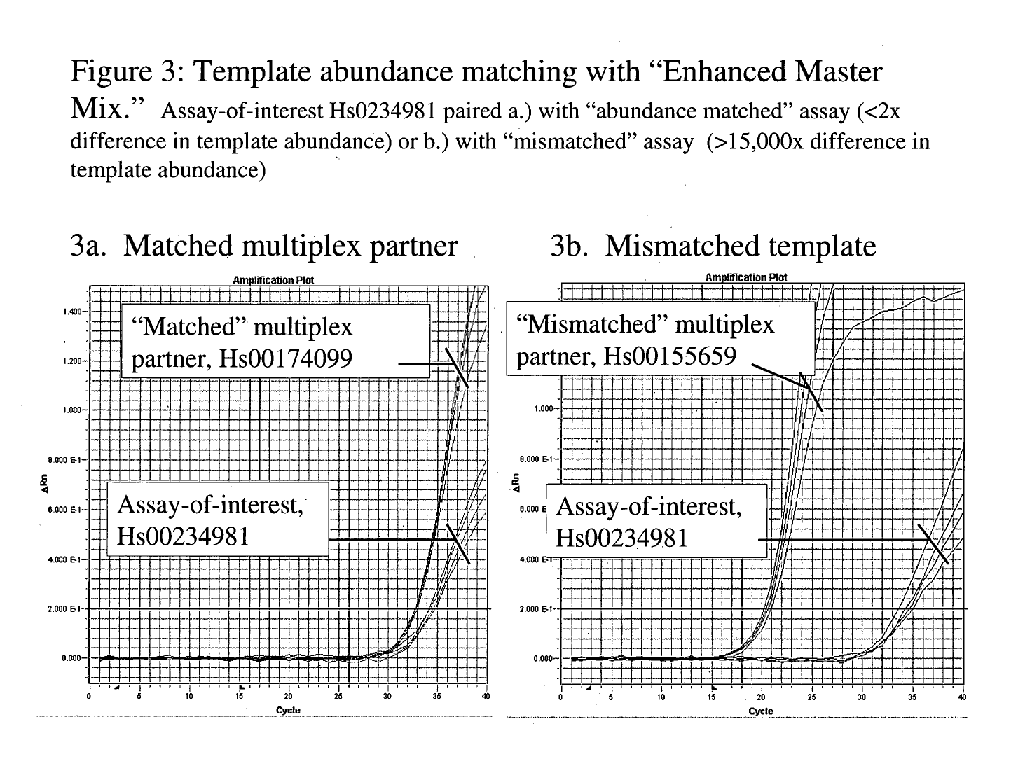

The multiplex real-time TaqMan PCR reactions of Example 2 were repeated, replacing the Universal Master Mix with the optimized Master Mix that was described in Example 1. The results for the reactions containing the similarly abundant target nucleic acid sequences Hs00174099 and Hs00234981 were similar to those obtained using Universal Master Mix (FIG. 3a, compared to FIG. 2a). The results for the reactions containing the differently abundant target nucleic acid sequences Hs00155659 and Hs00234981, however, were markedly different than those in Example 2 (FIG. 3b, compared to FIG. 2b). In the presence of optimized Master Mix, amplification of both the higher and the lower-abundance transcripts was observed (FIG. 3b). In the presence of Universal Master Mix, however, only amplification of the higher-abundance transcript was observed (FIG. 2b).

What is claimed is:

1. A method of amplifying at least two different target nucleic acid sequences in a sample comprising:
forming a plurality of different reaction compositions that each comprise a portion of the sample and at least two primer sets, wherein at least two of the primer sets are specific for a set of at least two different target nucleic acid sequences that are predicted to be present in similar abundance in the sample, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and
subjecting the plurality of different reaction compositions to at least one amplification reaction to amplify the sets of different target nucleic acid sequences.

2. The method of claim 1, wherein at least two of the different sets of target nucleic acid sequences comprise different target nucleic acid sequences that are in similar abundance in the sample.

3. The method of claim 1, wherein at least one of the at least two different sets of target nucleic acid sequences in one of the reaction compositions is at least 1000 times more abundant than at least one of the at least two different sets of target nucleic acid sequences of another different reaction composition.

4. The method of claim 1, wherein one reaction composition comprises at least one of the at least two different sets of target nucleic acid sequences at a copy number of 1 to 10 and another reaction composition comprises at least one of the at least two different sets of target nucleic acid sequences at a copy number of 1000 to 10,000.

5. The method of claim 1, wherein at least one of the different sets of target nucleic acid sequences comprises target nucleic acid sequences that are in similar abundance in the sample than other sets of target nucleic acid sequences.

6. The method of claim 1, wherein the forming of at least one of the reaction compositions further comprises combining with the sample and the at least two primer sets (a) polymerase at a minimum concentration of at least 0.015 U/μL, (b) dNTP's at a minimum concentration of at least 2 mM, and (c) magnesium at a minimum concentration of at least 1.5 mM.

7. The method of claim 6, wherein the forming of at least one of the reaction compositions comprises combining with the sample and the at least two primer sets at least one of the polymerase, the dNTP's, and the magnesium in a concentration greater than the minimum concentration.

8. The method of claim 1, wherein the amplification reaction is performed in a high-throughput assay system.

9. The method of claim 8, wherein the high-throughput assay system is at least one assay system selected from an Applied Biosystems plate-reader system, the ABI 7900 Micro Fluidic Card system, microfluidic systems that exploit the use of TaqMan probes, the Invader® system, the OpenArray™ system, systems including integrated fluidic circuits (Fluidigm), and other card reader microfluidic systems.

10. The method of claim 1, wherein amplification products of at least two different target nucleic acid sequences that are predicted to be present in similar abundance are detected in one of the reaction compositions after at least one amplification reaction, and wherein the concentration of the amplification product of one of the at least two different target nucleic acid sequences is within five to ten-fold of the concentration of the amplification product of another of the at least two different target nucleic acid sequences.

11. The method of claim 1, wherein amplification products of at least two different target nucleic acid sequences that are predicted to be present in similar abundance are detected in one of the reaction compositions after at least one amplification reaction, and wherein the concentration of the amplification product of one of the at least two different target nucleic acid sequences is 10 to 100-fold of the concentration of the amplification product of another of the at least two different target nucleic acid sequences.

12. The method of claim 1, wherein amplification products of at least two different target nucleic acid sequences that are predicted to be present in similar abundance are detected in one of the reaction compositions after at least one amplification reaction, and wherein the concentration of the amplification product of one of the at least two different target nucleic acid sequences is 100 to 1000-fold of the concentration of the amplification product of another of the at least two different target nucleic acid sequences.

13. A method of amplifying at least two different target nucleic acid sequences in a sample comprising:
forming a plurality of different reaction compositions that each comprise a portion of the sample and at least two primer sets, wherein at least two of the primer sets are specific for a set of at least two different target nucleic acid sequences that are present in similar abundance in the sample, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and
subjecting the plurality of different reaction compositions to at least one amplification reaction to amplify the sets of different target nucleic acid sequences.

14. The method of claim 13, wherein at least two of the different sets of target nucleic acid sequences comprise different target nucleic acid sequences that are in similar abundance in the sample.

15. The method of claim 13, wherein at least one of the at least two different sets of target nucleic acid sequences in one of the reaction compositions is at least 1000 times more abundant than at least one of the at least two different sets of target nucleic acid sequences of another different reaction composition.

16. The method of claim 13, wherein one reaction composition comprises at least one of the at least two different sets of target nucleic acid sequences at a copy number of 1 to 10 and another reaction composition comprises at least one of the at least two different sets of target nucleic acid sequences at a copy number of 1000 to 10,000.

17. The method of claim 13, wherein at least one of the different sets of target nucleic acid sequences comprises target nucleic acid sequences that are in similar abundance in the sample than other sets of target nucleic acid sequences.

18. The method of claim 13, wherein the forming of at least one of the reaction compositions further comprises combining with the sample and the at least two primer sets (a) polymerase at a minimum concentration of at least 0.015 U/μL, (b) dNTP's at a minimum concentration of at least 2 mM, and (c) magnesium at a minimum concentration of at least 1.5 mM.

19. The method of claim 18, wherein the forming of at least one of the reaction compositions comprises combining with the sample and the at least two primer sets at least one of the polymerase, the dNTP's, and the magnesium in a concentration greater than the minimum concentration.

20. The method of claim 13, wherein the amplification reaction is performed in a high-throughput assay system.

21. The method of claim 20, wherein the high-throughput assay system is selected from an Applied Biosystems plate-reader system, the ABI 7900 Micro Fluidic Card system, microfluidic systems that exploit the use of TaqMan probes, and other card reader microfluidic systems.

22. The method of claim 13, wherein amplification products of at least two different target nucleic acid sequences present in similar abundance are detected in one of the reaction compositions after at least one amplification reaction, and wherein the concentration of the amplification product of one of the at least two different target nucleic acid sequences is within five to ten-fold of the concentration of the amplification product of another of the at least two different target nucleic acid sequences.

23. The method of claim 13, wherein amplification products of at least two different target nucleic acid sequences that are present in similar abundance are detected in one of the reaction compositions after at least one amplification reaction, and wherein the concentration of the amplification product of one of the at least two different target nucleic acid sequences is 10 to 100-fold of the concentration of the amplification product of another of the at least two different target nucleic acid sequences.

24. The method of claim 13, wherein amplification products of at least two different target nucleic acid sequences that are present in similar abundance are detected in one of the reaction compositions after at least one amplification reaction, and wherein the concentration of the amplification product of one of the at least two different target nucleic acid sequences is 100 to 1000-fold of the concentration of the amplification product of another of the at least two different target nucleic acid sequences.

25. A method of amplifying at least two different target nucleic acid sequences in a sample comprising:
forming a plurality of different reaction compositions that each comprise a portion of the sample and at least two primer sets, wherein at least two of the primer sets are specific for a set of at least two different target nucleic acid sequences that are present at a copy number within 1000-fold of one another, wherein at least two of the primer sets of each of the plurality of different reaction compositions are different from primer sets in other reaction compositions of the plurality of different reaction compositions, such that different sets of target nucleic acid sequences are amplified in different reaction compositions during the at least one amplification reaction; and
subjecting the plurality of different reaction compositions to at least one amplification reaction to amplify the sets of different target nucleic acid sequences.


SEQUENCE LISTING (11 sequences)

SEQ ID NO: 1 - DNA, 27 bp, Artificial Sequence
  Amplification primer
ggctgtaactatctctgtgaagtgtga

SEQ ID NO: 2 - DNA, 23 bp, Artificial Sequence
  Amplification primer
tcctgggacacttctctgaaaga

SEQ ID NO: 3 - DNA, 17 bp, Artificial Sequence
  Amplification primer
agccgagccacatcgct

SEQ ID NO: 4 - DNA, 27 bp, Artificial Sequence
  Amplification primer
tggcaacaatatccactttaccagagt

SEQ ID NO: 5 - DNA, 145 bp, Artificial Sequence
  Hypothetical sequence
ggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccagga

SEQ ID NO: 6 - DNA, 122 bp, Artificial Sequence
  Hypothetical sequence
agccgagccacatcgctcagacaccatggggaaggtgaaggtcggagtcaacggatttggtcgtattgggcgcctggtcaccagggctgcttttaactctggtaaagtggatattgttgcca

SEQ ID NO: 7 - DNA, 19 bp, Artificial Sequence
  Reporter fluorochrome 6-carboxyfluorescein (“FAM”)
cctttaaggaaatgaatcc

SEQ ID NO: 8 - DNA, 15 bp, Artificial Sequence
  TaqMan probe
ccctggtgaccaggc

SEQ ID NO: 9 - DNA, 25 bp, Artificial Sequence
  Forward primer for HS00234981
cgtcagcggattatggattactatg

SEQ ID NO: 10 - DNA, 18 bp, Artificial Sequence
  Reverse primer for Hs00234981
acggaatggccccttttg

SEQ ID NO: 11 - DNA, 16 bp, Artificial Sequence
  Hs00234981 TaqMan probe
tgatgaagacaattcc